(19) United States
(12) Patent Application Publication
(10) Pub. No.: US 2011/0230485 A1
(43) Pub. Date: Sep. 22, 2011

(54) 6-PHENYL-PYRIMIDIN-4-YL-(PHENYLAMINE OR PHENOXY) DERIVATIVES USEFUL AS MODULATORS OF NICOTINIC ACETYLCHOLINE RECEPTORS

(75) Inventors: Antonio Nardi, Herzogenrath (DE); Jeppe Kejser Christensen, Glostrup (DK); Dan Peters, Malmo (SE)

(73) Assignee: NEUROSEARCH A/S, Ballerup (DK)

(21) Appl. No.: 13/122,983

(22) PCT Filed: Oct. 9, 2009
(86) PCT No.: PCT/EP09/63168

§ 371 Date: May 17, 2011

(30) Foreign Application Priority Data
Oct. 9, 2008 (DK) PA 2008 01421
May 12, 2009 (DK) PA 2009 00604

(51) Int. Cl. A61K 31/5377; C07D 239/48; C07D 401/12; C07D 403/12; C07D 405/12; C07D 239/47; C07D 413/12; A61K 31/505; A61K 31/506; A61P 25/22; A61P 25/00; A61P 25/28; A61P 25/16; A61P 25/24; A61P 25/18; A61P 3/04; A61P 25/20; A61P 25/14; A61P 25/08; A61P 43/00; A61P 9/12; A61P 9/06; A61P 21/00; A61P 1/12; A61P 11/06; A61P 15/00; A61P 5/00; A61P 25/04; A61P 25/06; A61P 29/00; A61P 17/00; A61P 17/10; A61P 25/30; A61P 25/36

(52) U.S. Cl. 514/235.8; 544/326; 544/328; 544/327; 544/319; 544/123; 514/256; 514/269

(57) ABSTRACT

This invention relates to novel 6-phenyl-phymidin-4-yl-(phenylannine or phenoxy) derivatives, which are found to be modulators of the nicotinic acetylcholine receptors. Due to their pharmacological profile the compounds of the invention may be useful for the treatment of diseases or disorders as diverse as those related to the cholinergic system of the central nervous system (CNS), the peripheral nervous system (PNS), diseases or disorders related to smooth muscle contraction, endocrine diseases or disorders, diseases or disorders related to neuro-degeneration, diseases or disorders related to inflammation, pain, and withdrawal symptoms caused by the termination of abuse of chemical substances.

### TECHNICAL FIELD

This invention relates to novel 6-phenyl-pyrimidin-4-yl-(phenylamine or phenoxy) derivatives, which are found to be modulators of the nicotinic acetylcholine receptors. Due to their pharmacological profile the compounds of the invention may be useful for the treatment of diseases or disorders as diverse as those related to the cholinergic system of the central nervous system (CNS), the peripheral nervous system (PNS), diseases or disorders related to smooth muscle contraction, endocrine diseases or disorders, diseases or disorders related to neuro-degeneration, diseases or disorders related to inflammation, pain, and withdrawal symptoms caused by the termination of abuse of chemical substances.

### BACKGROUND ART

The endogenous cholinergic neurotransmitter, acetylcholine, exert its biological effect via two types of cholinergic receptors, the muscarinic Acetyl Choline Receptors (mAChR) and the nicotinic Acetyl Choline Receptors (nAChR).

As it is well established that muscarinic acetylcholine receptors dominate quantitatively over nicotinic acetylcholine receptors in the brain area important to memory and cognition, and much research aimed at the development of agents for the treatment of memory related disorders have focused on the synthesis of muscarinic acetylcholine receptor modulators.

Recently, however, an interest in the development of nAChR modulators has emerged. Several diseases are associated with degeneration of the cholinergic system i.e. senile dementia of the Alzheimer type, vascular dementia and cognitive impairment due to the organic brain damage disease related directly to alcoholism.

WO 99001439 describes aryl- and arylamino-substituted heterocyclic compounds useful as corticotropin releasing hormone antagonists. However, the 6-phenyl-pyrimidin-4-yl-(phenylamine or phenoxy) derivatives of the present invention, or their use as modulators of the nicotinic acetylcholine receptors, are not suggested.

WO 2005 009977 describes substituted pyrimidin-4-yl-amine analogs useful as vanilloid receptor ligands. However, the 6-phenyl-pyrimidin-4-yl-(phenylamine or phenoxy) derivatives of the present invention, or their use as modulators of the nicotinic acetylcholine receptors, are not suggested.

Hauser et al. [Hauser D R J.; Scior T; Domeyer D M.; Kammerer B; Laufer S A; Journal of Medicinal Chemistry 2007 50 (9) 2060-2066] describes the synthesis, biological testing and binding mode prediction of 6,9-Diarylpurin-8-ones useful as p38 MAP Kinase Inhibitors. However, the 6-phenyl-pyrimidin-4-yl-(phenylamine or phenoxy) derivatives of the present invention, or their use as modulators of the nicotinic acetylcholine receptors, are not suggested.

### SUMMARY OF THE INVENTION

The present invention is devoted to the provision novel modulators of the nicotinic receptors, which modulators are useful for the treatment of diseases or disorders related to the cholinergic receptors, and in particular the nicotinic acetylcholine α7 receptor subtype.

The compounds of the invention may also be useful as diagnostic tools or monitoring agents in various diagnostic methods, and in particular for in vivo receptor imaging (neuroimaging), and they may be used in labelled or unlabelled form.

In its first aspect the invention provides 6-phenyl-pyrimidin-4-yl-(phenylamine or phenoxy) derivatives represented by Formula I

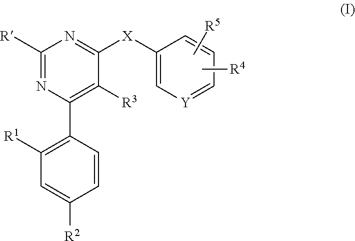

a stereoisomer thereof or a mixture of its stereoisomers, or a pharmaceutically acceptable salt thereof, wherein

X represents O or NH;

Y represents CH or N;

R′ represents hydrogen or alkyl;

R¹ and R², independently of each other, represent a substituent selected from the group consisting of hydrogen, halo, trifluoromethyl, trifluoromethoxy, nitro, cyano and hydroxy;

R³ represents amino or nitro; and

R⁴ and R⁵, independently of each other, represent hydrogen, halo, trifluoromethyl, hydroxy, alkoxy, thioalkoxy, cycloalkoxy, alkyl, cycloalkyl, sulfamoyl, piperidinyl, morpholinyl, or pyridinyl; or R⁴ and R⁵ together with the phenyl ring to which they are attached form a methylenedioxy group or ethylenedioxy group; or R⁴ and R⁵ together with the phenyl ring to which they are attached form a bicyclic carbocyclic ring selected from naphthyl and tetrahydronaphthalenyl; or R⁴ and R⁵ together with the phenyl ring to which they are attached form a bicyclic heterocyclic ring selected from indolyl, indazolyl, quinolinyl and isoquinolinyl.

In a second aspect the invention provides pharmaceutical compositions comprising a therapeutically effective amount of the 6-phenyl-pyrimidin-4-yl-(phenylamine or phenoxy) derivative of the invention, or a pharmaceutically acceptable addition salt thereof, together with at least one pharmaceutically acceptable carrier or diluent.

Viewed from another aspect the invention relates to the use of the 6-phenyl-pyrimidin-4-yl-(phenylamine or phenoxy) derivative of the invention, or a pharmaceutically acceptable addition salt thereof, for the manufacture of pharmaceutical compositions/medicaments for the treatment, prevention or alleviation of a disease or a disorder or a condition of a mammal, including a human, which disease, disorder or condition is responsive to modulation of cholinergic receptors.

In yet another aspect the invention provides a method for treatment, prevention or alleviation of diseases, disorders or conditions of a living animal body, including a human, which disorder, disease or condition is responsive to modulation of cholinergic receptors, and which method comprises the step of administering to such a living animal body in need thereof a therapeutically effective amount of the 6-phenyl-pyrimidin-4-yl-(phenylamine or phenoxy) derivative of the invention.

Other objects of the invention will be apparent to the person skilled in the art from the following detailed description and examples.

### DETAILED DISCLOSURE OF THE INVENTION

### 6-Phenyl-pyrimidin-4-yl-(phenylamine or phenoxy) derivatives

In its first aspect the invention provides 6-phenyl-pyrimidin-4-yl-(phenylamine or phenoxy) derivatives represented by Formula I

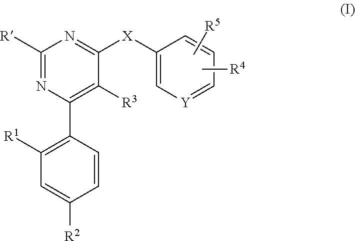

a stereoisomer thereof or a mixture of its stereoisomers, or a pharmaceutically acceptable salt thereof, wherein

X represents O or NH;

Y represents CH or N;

R′ represents hydrogen or alkyl;

R¹ and R², independently of each other, represent a substituent selected from the group consisting of hydrogen, halo, trifluoromethyl, trifluoromethoxy, nitro, cyano and hydroxy;

R³ represents amino or nitro; and

R⁴ and R⁵, independently of each other, represent hydrogen, halo, trifluoromethyl, hydroxy, alkoxy, thioalkoxy, cycloalkoxy, alkyl, cycloalkyl, sulfamoyl, piperidinyl, morpholinyl, or pyridinyl; or R⁴ and R⁵ together with the phenyl ring to which they are attached form a methylenedioxy group or ethylenedioxy group; or R⁴ and R⁵ together with the phenyl ring to which they are attached form a bicyclic carbocyclic ring selected from naphthyl and tetrahydronaphthalenyl; or R⁴ and R⁵ together with the phenyl ring to which they are attached form a bicyclic heterocyclic ring selected from indolyl, indazolyl, quinolinyl and isoquinolinyl.

In a more preferred embodiment, the invention provides a 6-phenyl-pyrimidin-4-yl-(phenylamine or phenoxy) derivative represented by Formula Ia

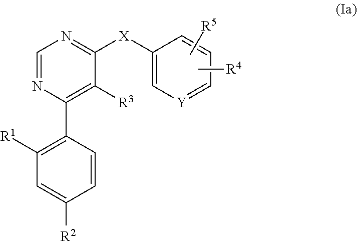

a stereoisomer thereof or a mixture of its stereoisomers, or a pharmaceutically acceptable salt thereof, wherein

X represents O or NH;

Y represents CH or N;

R¹ and R², independently of each other, represent a substituent selected from the group consisting of hydrogen, halo, trifluoromethyl, trifluoromethoxy, nitro, cyano and hydroxy;

R³ represents amino or nitro; and

R⁴ and R⁵, independently of each other, represent hydrogen, hydroxy, alkoxy, sulfamoyl or pyridinyl; or R⁴ and R⁵ together with the phenyl ring to which they are attached form a bicyclic heterocyclic ring selected from indolyl and indazolyl.

In another more preferred embodiment, the invention provides a 6-phenyl-pyrimidin-4-yl-(phenylamine or phenoxy) derivative represented by Formula Ib

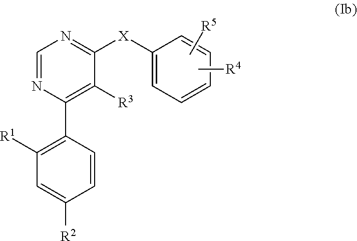

a stereoisomer thereof or a mixture of its stereoisomers, or a pharmaceutically acceptable salt thereof, wherein

X represents O or NH; and

R¹, R², R³, R⁴ and R⁵, are as defined above. In a third more preferred embodiment, the invention provides a 6-phenyl-pyrimidin-4-yl-(phenylamine or phenoxy) derivative represented by Formula Ic

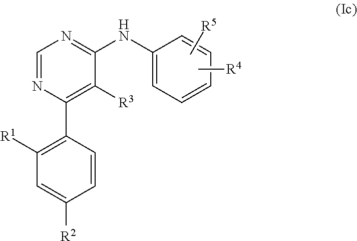

a stereoisomer thereof or a mixture of its stereoisomers, or a pharmaceutically acceptable salt thereof, wherein

R¹ and R², independently of each other, represent a substituent selected from the group consisting of hydrogen, halo, trifluoromethyl, trifluoromethoxy, nitro, cyano and hydroxy;

R³ represents amino or nitro; and

R⁴ and R⁵, independently of each other, represent hydrogen, hydroxy, alkoxy, sulfamoyl or pyridinyl; or R⁴ and R⁵ together with the phenyl ring to which they are attached form a bicyclic heterocyclic ring selected from indolyl and indazolyl.

In a third more preferred embodiment, the invention provides a 6-phenyl-pyrimidin-4-yl-(phenylamine or phenoxy) derivative represented by Formula Id

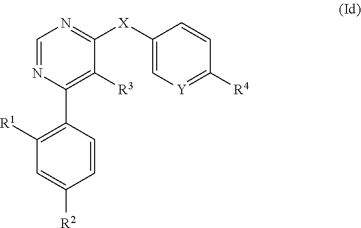

a stereoisomer thereof or a mixture of its stereoisomers, or a pharmaceutically acceptable salt thereof, wherein

X represents O or NH;

Y represents CH or N;

R¹, R² and R³ are as defined above; and

R⁴ represents hydroxy, alkoxy, sulfamoyl or pyridinyl.

In a fifth more preferred embodiment, the invention provides a 6-phenyl-pyrimidin-4-yl-(phenylamine or phenoxy) derivative represented by Formula Ie

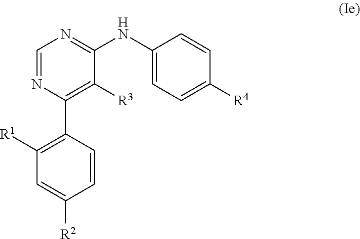

a stereoisomer thereof or a mixture of its stereoisomers, or a pharmaceutically acceptable salt thereof, wherein

R¹, R² and R³ are as defined above; and

R⁴ represents hydroxy, alkoxy, sulfamoyl or pyridinyl.

In another preferred embodiment, the 6-phenyl-pyrimidin-4-yl-(phenylamine or phenoxy) derivative of the invention is a compound of Formula I, Ia, Ib, Ic or Id, a stereoisomer thereof or a mixture of its stereoisomers, or a pharmaceutically acceptable salt thereof, wherein X represents O or NH.

In a more preferred embodiment, X represents O.

In another more preferred embodiment, X represents NH.

In a third preferred embodiment, the 6-phenyl-pyrimidin-4-yl-(phenylamine or phenoxy) derivative of the invention is a compound of Formula I, Ia, Ib, Ic or Id, a stereoisomer thereof or a mixture of its stereoisomers, or a pharmaceutically acceptable salt thereof, wherein Y represents CH or N.

In a more preferred embodiment, Y represents CH.

In another more preferred embodiment, Y represents N.

In a fourth preferred embodiment, the 6-phenyl-pyrimidin-4-yl-(phenylamine or phenoxy) derivative of f the invention is a compound of Formula I, Ia, Ib, Ic or Id, a stereoisomer thereof or a mixture of its stereoisomers, or a pharmaceutically acceptable salt thereof, wherein R′ represents hydrogen or alkyl.

In a more preferred embodiment, R′ represents hydrogen.

In another more preferred embodiment, R′ represents alkyl, and in particular methyl.

In a fifth preferred embodiment, the 6-phenyl-pyrimidin-4-yl-(phenylamine or phenoxy) derivative of the invention is a compound of Formula I, Ia, Ib, Ic or Id, a stereoisomer thereof or a mixture of its stereoisomers, or a pharmaceutically acceptable salt thereof, wherein R¹ and R², independently of each other, represent a substituent selected from the group consisting of hydrogen, halo, trifluoromethyl, trifluoromethoxy, nitro, cyano and hydroxy.

In a more preferred embodiment, R¹ and R², independently of each other, represent a substituent selected from the group consisting of halo, trifluoromethyl, trifluoromethoxy, nitro, cyano and hydroxy.

In another more preferred embodiment, R¹ and R², independently of each other, represent a substituent selected from the group consisting of halo, trifluoromethyl, trifluoromethoxy, nitro and cyano.

In a third more preferred embodiment, R¹ and R², independently of each other, represent a substituent selected from the group consisting of halo, and in particular fluoro, and trifluoromethyl.

In a fourth more preferred embodiment, R¹ represents hydrogen or halo, and in particular fluoro; and R² represents trifluoromethyl, trifluoromethoxy, nitro or cyano.

In a fifth more preferred embodiment, R¹ represents halo, and in particular fluoro; and R² represents trifluoromethyl, trifluoromethoxy, nitro or cyano.

In a sixth more preferred embodiment, R¹ represents halo, and in particular fluoro; and R² represents trifluoromethyl.

In a sixth preferred embodiment, the 6-phenyl-pyrimidin-4-yl-(phenylamine or phenoxy) derivative of the invention is a compound of Formula I, Ia, Ib, Ic or Id, a stereoisomer thereof or a mixture of its stereoisomers, or a pharmaceutically acceptable salt thereof, wherein R³ represents amino or nitro.

In a more preferred embodiment, R³ represents amino.

In another more preferred embodiment, R³ represents nitro.

In a seventh preferred embodiment, the 6-phenyl-pyrimidin-4-yl-(phenylamine or phenoxy) derivative of the invention is a compound of Formula I, Ia, Ib, Ic or Id, a stereoisomer thereof or a mixture of its stereoisomers, or a pharmaceutically acceptable salt thereof, wherein R⁴ and R⁵, independently of each other, represent hydrogen, halo, trifluoromethyl, hydroxy, alkoxy, thioalkoxy, cycloalkoxy, alkyl, cycloalkyl, sulfamoyl, piperidinyl, morpholinyl, or pyridinyl.

In a more preferred embodiment, R⁴ and R⁵, independently of each other, represent hydrogen, hydroxy, alkoxy, sulfamoyl or pyridinyl; or R⁴ and R⁵ together with the phenyl ring to which they are attached form a bicyclic heterocyclic ring selected from indolyl and indazolyl.

In another more preferred embodiment, R⁴ and R⁵, independently of each other, represent hydrogen, halo, trifluoromethyl, hydroxy, alkoxy, thioalkoxy, cycloalkoxy, alkyl, cycloalkyl, sulfamoyl, piperidinyl, morpholinyl, or pyridinyl.

In a third more preferred embodiment, one of R⁴ and R⁵ represents hydrogen; and the other of R⁴ and R⁵ represents hydroxy, halo, alkoxy, thioalkoxy, cycloalkoxy, alkyl, cycloalkyl, sulfamoyl, piperidinyl, morpholinyl, or pyridinyl.

In a fourth more preferred embodiment, one of R⁴ and R⁵ represents hydrogen; and the other of R⁴ and R⁵ represents hydroxy, alkoxy, sulfamoyl or pyridinyl.

In a fifth more preferred embodiment, one of R⁴ and R⁵ represents hydrogen; and the other of R⁴ and R⁵ represents hydroxy.

In a sixth more preferred embodiment, one of R⁴ and R⁵ represents hydrogen; and the other of R⁴ and R⁵ represents alkoxy, and in particular methoxy.

In a seventh more preferred embodiment, one of R⁴ and R⁵ represents hydrogen; and the other of R⁴ and R⁵ represents sulfamoyl.

In an eight more preferred embodiment, one of R⁴ and R⁵ represents hydrogen; and the other of R⁴ and R⁵ represents pyridinyl.

In a ninth more preferred embodiment, one of R⁴ and R⁵ represents halo, and in particular fluoro or chloro, or alkoxy, and in particular methoxy; and the other of R⁴ and R⁵ represents halo, and in particular fluoro or chloro, trifluoromethyl, alkoxy, and in particular methoxy.

In a tenth more preferred embodiment, the 6-phenyl-pyrimidin-4-yl-(phenylamine or phenoxy) derivative of the invention is a compound of Formula Ic or 1d, a stereoisomer thereof or a mixture of its stereoisomers, or a pharmaceutically acceptable salt thereof, wherein

R⁴ represents halo, trifluoromethyl, hydroxy, alkoxy, cycloalkoxy, alkyl, sulfamoyl or pyridinyl.

In an eleventh more preferred embodiment, the 6-phenyl-pyrimidin-4-yl-(phenylamine or phenoxy) derivative of the invention is a compound of Formula Ic or 1d, a stereoisomer thereof or a mixture of its stereoisomers, or a pharmaceutically acceptable salt thereof, wherein R⁴ represents hydroxy, alkoxy, sulfamoyl or pyridinyl.

In a twelfth more preferred embodiment, the 6-phenyl-pyrimidin-4-yl-(phenylamine or phenoxy) derivative of the invention is a compound of Formula Ic or 1d, a stereoisomer thereof or a mixture of its stereoisomers, or a pharmaceutically acceptable salt thereof, wherein R⁴ represents hydroxy.

In a thirteenth more preferred embodiment, the 6-phenyl-pyrimidin-4-yl-(phenylamine or phenoxy) derivative of the invention is a compound of Formula Ic or 1d, a stereoisomer thereof or a mixture of its stereoisomers, or a pharmaceutically acceptable salt thereof, wherein R⁴ represents alkoxy, and in particular methoxy. p In a fourteenth more preferred embodiment, the 6-phenyl-pyrimidin-4-yl-(phenylamine or phenoxy) derivative of the invention is a compound of Formula Ic or 1d, a stereoisomer thereof or a mixture of its stereoisomers, or a pharmaceutically acceptable salt thereof, wherein R⁴ represents sulfamoyl.

In a fifteenth more preferred embodiment, the 6-phenyl-pyrimidin-4-yl-(phenylamine or phenoxy) derivative of the invention is a compound of Formula Ic or 1d, a stereoisomer thereof or a mixture of its stereoisomers, or a pharmaceutically acceptable salt thereof, wherein R⁴ represents pyridinyl, and in particular pyridin-3-yl.

In an eight preferred embodiment, the 6-phenyl-pyrimidin-4-yl-(phenylamine or phenoxy) derivative of the invention is a compound of Formula I, Ia, Ib, Ic or Id, a stereoisomer thereof or a mixture of its stereoisomers, or a pharmaceutically acceptable salt thereof, wherein R⁴ and R⁵ together with the phenyl ring to which they are attached form a methylenedioxy group or ethylenedioxy group.

In a more preferred embodiment, R⁴ and R⁵ together with the phenyl ring to which they are attached form a methylenedioxy group.

In another more preferred embodiment, R⁴ and R⁵ together with the phenyl ring to which they are attached form a ethylenedioxy group.

In a ninth preferred embodiment, the 6-phenyl-pyrimidin-4-yl-(phenylamine or phenoxy) derivative of the invention is a compound of Formula I, Ia, Ib, Ic or Id, a stereoisomer thereof or a mixture of its stereoisomers, or a pharmaceutically acceptable salt thereof, wherein R⁴ and R⁵ together with the phenyl ring to which they are attached form a bicyclic carbocyclic ring selected from naphthyl and tetrahydronaphthalenyl.

In a more preferred embodiment, R⁴ and R⁵ together with the phenyl ring to which they are attached form a naphthyl ring.

In another more preferred embodiment, R⁴ and R⁵ together with the phenyl ring to which they are attached form a tetrahydronaphthalenyl ring, and in particular a 5,6,7,8-tetrahydro-naphthalen-2-yl ring.

In a tenth preferred embodiment, the 6-phenyl-pyrimidin-4-yl-(phenylamine or phenoxy) derivative of the invention is a compound of Formula I, Ia, Ib, Ic or Id, a stereoisomer thereof or a mixture of its stereoisomers, or a pharmaceutically acceptable salt thereof, wherein R⁴ and R⁵ together with the phenyl ring to which they are attached form a bicyclic heterocyclic ring selected from indolyl, indazolyl, quinolinyl and isoquinolinyl.

In a more preferred embodiment, R⁴ and R⁵ together with the phenyl ring to which they are attached form an indolyl group, and in particular a 1H-indol-5-yl group.

In another more preferred embodiment, R⁴ and R⁵ together with the phenyl ring to which they are attached form an indazolyl group, and in particular a1H-indazol-5-yl group.

In a third more preferred embodiment, R⁴ and R⁵ together with the phenyl ring to which they are attached form a quinolinyl ring, and in particular a quinolin-8-yl ring.

In a fourth more preferred embodiment, R⁴ and R⁵ together with the phenyl ring to which they are attached form an isoquinolinyl ring, and in particular an isoquinolin-5-yl ring.

In a most preferred embodiment, the 6-phenyl-pyrimidin-4-yl-(phenylamine or phenoxy) derivative of the invention is

[6-(2-Fluoro-4-trifluoromethyl-phenyl)-5-nitro-pyrimidin-4-yl]-(4-methoxy-phenyl)-amine;

4-[6-(2-Fluoro-4-trifluoromethyl-phenyl)-5-nitro-pyrimidin-4-ylamino]-phenol;

6-(2-Fluoro-4-trifluoromethyl-phenyl)-N*4*-(4-methoxy-phenyl)-pyrimidine-4,5-diamine;

4-[5-Amino-6-(2-fluoro-4-trifluoromethyl-phenyl)-pyrimidin-4-ylamino]-phenol;

[6-(2-Fluoro-4-trifluoromethyl-phenyl)-5-nitro-pyrimidin-4-yl]-(6-methoxy-pyridin-3-yl)-amine;

6-(2-Fluoro-4-trifluoromethyl-phenyl)-N*4*-(6-methoxy-pyridin-3-yl)-pyrimidine-4,5-diamine;

4-[6-(2-Fluoro-4-trifluoromethyl-phenyl)-5-nitro-pyrimidin-4-ylamino]-benzenesulfonamide;

4-[5-Amino-6-(2-fluoro-4-trifluoromethyl-phenyl)-pyrimidin-4-ylamino]-benzenesulfonamide;

[6-(2-Fluoro-4-trifluoromethyl-phenyl)-5-nitro-pyrimidin-4-yl]-(1H-indol-5-yl)-amine;

6-(2-Fluoro-4-trifluoromethyl-phenyl)-N*4*-(1H-indol-5-yl)-pyrimidine-4,5-diamine;

5-[5-Amino-6-(2-fluoro-4-trifluoromethyl-phenyl)-pyrimidin-4-ylamino]-pyridin-2-ol;

6-(2-Fluoro-4-trifluoromethyl-phenyl)-N*4*-(1H-indazol-5-yl)-pyrimidine-4,5-diamine;

N*4*-Benzo[1,3]dioxol-5-yl-6-(2-fluoro-4-trifluoromethyl-phenyl)-pyrimidine-4,5-diamine;

N*4*-(4-Cyclopropylmethoxy-phenyl)-6-(2-fluoro-4-trifluoromethyl-phenyl)-pyrimidine-4,5-diamine;

6-(2-Fluoro-4-trifluoromethyl-phenyl)-2-methyl-N*4*-naphthalen-1-yl-pyrimidine-4,5-diamine;

6-(2-Fluoro-4-trifluoromethyl-phenyl)-N*4*-(4-methoxy-phenyl)-2-methyl-pyrimidine-4,5-diamine;

N*4*-(2,4-Difluoro-phenyl)-6-(2-fluoro-4-trifluoromethyl-phenyl)-2-methyl-pyrimidine-4,5-diamine;

N*4*-(4-Fluoro-phenyl)-6-(2-fluoro-4-trifluoromethyl-phenyl)-2-methyl-pyrimidine-4,5-diamine;

N*4*-(2-Fluoro-phenyl)-6-(2-fluoro-4-trifluoromethyl-phenyl)-2-methyl-pyrimidine-4,5-diamine;

N*4*-(2,4-Dichloro-phenyl)-6-(2-fluoro-4-trifluoromethyl-phenyl)-2-methyl-pyrimidine-4,5-diamine;

N*4*-Benzo[1,3]dioxol-5-yl-6-(2-fluoro-4-trifluoromethyl-phenyl)-2-methyl-pyrimidine-4,5-diamine;

N*4*-(3,5-Dimethoxy-phenyl)-6-(2-fluoro-4-trifluoromethyl-phenyl)-2-methyl-pyrimidine-4,5-diamine;

N*4*-(4-Chloro-3-trifluoromethyl-phenyl)-6-(2-fluoro-4-trifluoromethyl-phenyl)-2-methyl-pyrimidine-4,5-diamine;

N*4*-(3-Fluoro-phenyl)-6-(2-fluoro-4-trifluoromethyl-phenyl)-2-methyl-pyrimidine-4,5-diamine;

N*4*-(4-Cyclohexyl-phenyl)-6-(2-fluoro-4-trifluoromethyl-phenyl)-2-methyl-pyrimidine-4,5-diamine;

N*4*-(4-Ethoxy-phenyl)-6-(2-fluoro-4-trifluoromethyl-phenyl)-2-methyl-pyrimidine-4,5-diamine;

6-(2-Fluoro-4-trifluoromethyl-phenyl)-2-methyl-N*4*-p-tolyl-pyrimidine-4,5-diamine;

6-(2-Fluoro-4-trifluoromethyl-phenyl)-N*4*-isoquinolin-5-yl-2-methyl-pyrimidine-4,5-diamine;

N*4*-(2,3-Dihydro-benzo[1,4]dioxin-6-yl)-6-(2-fluoro-4-trifluoromethyl-phenyl)-2-methyl-pyrimidine-4,5-diamine;

N*4*-(3,4-Dimethoxy-phenyl)-6-(2-fluoro-4-trifluoromethyl-phenyl)-2-methyl-pyrimidine-4,5-diamine;

6-(2-Fluoro-4-trifluoromethyl-phenyl)-N*4*-(3-methoxy-phenyl)-2-methyl-pyrimidine-4,5-diamine;

6-(2-Fluoro-4-trifluoromethyl-phenyl)-2-methyl-N*4*-(3-methylsulfanyl-phenyl)-pyrimidine-4,5-diamine;

N*4*-(2,4-Dimethoxy-phenyl)-6-(2-fluoro-4-trifluoromethyl-phenyl)-2-methyl-pyrimidine-4,5-diamine;

6-(2-Fluoro-4-trifluoromethyl-phenyl)-2-methyl-N*4*-naphthalen-2-yl-pyrimidine-4,5-diamine;

4-(2-Fluoro-4-trifluoromethyl-phenyl)-6-(4-methoxy-phenoxy)-5-nitro-pyrimidine;

4-(2-Fluoro-4-trifluoromethyl-phenyl)-6-(4-methoxy-phenoxy)-pyrimidin-5-ylamine;

4-[5-Amino-6-(2-fluoro-4-trifluoromethyl-phenyl)-pyrim id in-4-yloxy]-phenol;

4-(2-Fluoro-4-trifluoromethyl-phenyl)-2-methyl-6-(2-propyl-phenoxy)-pyrimidin-5-ylamine;

4-(2-Fluoro-4-trifluoromethyl-phenyl)-2-methyl-6-(2-morpholin-4-yl-phenoxy)-pyrimidin-5-ylamine;

4-(2-Fluoro-4-trifluoromethyl-phenyl)-2-methyl-6-(quinolin-8-yloxy)-pyrimidin-5-ylamine; 4-(2-Fluoro-4-trifluoromethyl-phenyl)-2-methyl-6-(5,6,7,8-tetrahydro-naphthalen-2-yloxy)-pyrimidin-5-ylamine;

4-(4-Fluoro-phenoxy)-6-(2-fluoro-4-trifluoromethyl-phenyl)-2-methyl-pyrimidin-5-ylamine;

4-(Benzo[1,3]dioxol-5-yloxy)-6-(2-fluoro-4-trifluoromethyl-phenyl)-2-methyl-pyrimidin-5-ylamine;

4-(2-Chloro-4-methoxy-phenoxy)-6-(2-fluoro-4-trifluoromethyl-phenyl)-2-methyl-pyrimidin-5-ylamine;

4-(3-Fluoro-phenoxy)-6-(2-fluoro-4-trifluoromethyl-phenyl)-2-methyl-pyrimidin-5-ylamine;

4-(2-Chloro-4-trifluoromethyl-phenoxy)-6-(2-fluoro-4-trifluoromethyl-phenyl)-2-methyl-pyrimidin-5-ylamine;

4-(2-Fluoro-4-trifluoromethyl-phenyl)-6-(4-methoxy-phenoxy)-2-methyl-pyrimidin-5-ylamine;

4-(3-Chloro-5-methoxy-phenoxy)-6-(2-fluoro-4-trifluoromethyl-phenyl)-2-methyl-pyrimidin-5-ylamine;

4-(3-tert-Butyl-phenoxy)-6-(2-fluoro-4-trifluoromethyl-phenyl)-2-methyl-pyrimidin-5-ylamine;

4-(4-tert-Butyl-phenoxy)-6-(2-fluoro-4-trifluoromethyl-phenyl)-2-methyl-pyrimidin-5-ylamine;

4-(2-Fluoro-4-trifluoromethyl-phenyl)-2-methyl-6-(2-piperidin-1-yl-phenoxy)-pyrimidin-5-ylamine;

4-(2-Fluoro-phenoxy)-6-(2-fluoro-4-trifluoromethyl-phenyl)-2-methyl-pyrimidin-5-ylamine; or

4-(2-Fluoro-4-trifluoromethyl-phenyl)-2-methyl-6-phenoxy-pyrimidin-5-ylamine;

a stereoisomer thereof or a mixture of its stereoisomers, or a pharmaceutically acceptable salt thereof.

Any combination of two or more of the embodiments described herein is considered within the scope of the present invention.

### Definition of Substituents

In the context of this invention halo represents fluoro, chloro, bromo or iodo.

In the context of this invention an alkyl group designates a univalent saturated, straight or branched hydrocarbon chain. The hydrocarbon chain preferably contain of from one to eighteen carbon atoms (C₁₋₁₈-alkyl), more preferred of from one to six carbon atoms (C₁₋₆-alkyl; lower alkyl), including pentyl, isopentyl, neopentyl, hexyl and isohexyl. In a preferred embodiment alkyl represents a C₁₋₄-alkyl group, including butyl, isobutyl, secondary butyl, and tertiary butyl. In another preferred embodiment of this invention alkyl represents a C₁₋₃-alkyl group, which may in particular be methyl, ethyl, propyl or isopropyl.

In the context of this invention an alkoxy group designates an “alkyl-O—” group, wherein alkyl designates a univalent saturated, straight or branched hydrocarbon chain. The hydrocarbon chain preferably contain of from one to eighteen carbon atoms (C₁₋₁₈-alkyl), more preferred of from one to six carbon atoms (C₁₋₆-alkyl; lower alkyl), including pentyl, isopentyl, neopentyl, tertiary pentyl, hexyl and isohexyl. Preferred alkoxy groups of the invention include methoxy, ethoxy and isopropoxy.

In the context of this invention an thioalkoxy group designates an “alkyl-S-” group, wherein alkyl is as defined above. Examples of preferred thioalkoxy groups of the invention include methylthio (thiomethoxy or methylsulfanyl), and ethylthio (thioethoxy or ethylsulfanyl).

In the context of this invention a cycloalkyl group designates a cyclic alkyl group, preferably containing of from three to seven carbon atoms (C₃₋₇-cycloalkyl), including cyclopropyl, cyclobutyl, cyclopentyl, cyclohexyl and cycloheptyl.

In the context of this invention a cycloalkoxy group designates a “cycloalkyl-O—” group, wherein cycloalkyl is as defined above. A preferred alkoxy group of the invention is cyclopropylmethoxy and cyclopropoxy.

### Pharmaceutically Acceptable Salts

The 6-phenyl-pyrimidin-4-yl-(phenylamine or phenoxy) derivative of the invention may be provided in any form suitable for the intended administration. Suitable forms include pharmaceutically (i.e. physiologically) acceptable salts, and pre- or prodrug forms of the compound of the invention.

Examples of pharmaceutically acceptable addition salts include, without limitation, the non-toxic inorganic and organic acid addition salts such as the hydrochloride, the hydrobromide, the nitrate, the perchlorate, the phosphate, the sulphate, the formate, the acetate, the aconate, the ascorbate, the benzenesulphonate, the benzoate, the cinnamate, the citrate, the embonate, the enantate, the fumarate, the glutamate, the glycolate, the lactate, the maleate, the malonate, the mandelate, the methanesulphonate, the naphthalene-2-sulphonate derived, the phthalate, the salicylate, the sorbate, the stearate, the succinate, the tartrate, the toluene-p-sulphonate, and the like. Such salts may be formed by procedures well known and described in the art.

Metal salts of a 6-phenyl-pyrimidin-4-yl-(phenylamine or phenoxy) derivative of the invention include alkali metal salts, such as the sodium salt of a compound of the invention containing a carboxy group.

### Steric Isomers

It will be appreciated by those skilled in the art that the 6-phenyl-pyrimidin-4-yl-(phenylamine or phenoxy) derivatives of the present invention may exist in different stereo isomeric forms, including enantiomers, diastereomers, as well as geometric isomers (cis-trans isomers). The invention includes all such stereoisomers and any mixtures thereof including racemic mixtures.

Racemic forms can be resolved into the optical antipodes by known methods and techniques. One way of separating the enantiomeric compounds (including enantiomeric intermediates) is—in the case the compound being a chiral acid by use of an optically active amine, and liberating the diastereomeric, resolved salt by treatment with an acid. Another method for resolving racemates into the optical antipodes is based upon chromatography on an optical active matrix. Racemic compounds of the present invention can thus be resolved into their optical antipodes, e.g., by fractional crystallisation of D- or L-(tartrates, mandelates, or camphorsulphonate) salts for example.

Additional methods for the resolving the optical isomers are known in the art. Such methods include those described by Jaques J, Collet A, & Wilen S in “Enantiomers, Racemates, and Resolutions”, John Wiley and Sons, New York (1981).

Optical active compounds can also be prepared from optically active starting materials or intermediates.

Methods of Producing 6-phenyl-pyrimidin-4-yl-(phenylamine or phenoxy) Derivatives

The 6-phenyl-pyrimidin-4-yl-(phenylamine or phenoxy) derivative of the invention may be prepared by conventional methods for chemical synthesis, e.g. those described in the working examples. The starting materials for the processes described in the present application are known or may readily be prepared by conventional methods from commercially available chemicals.

Also one compound of the invention can be converted to another compound of the invention using conventional methods.

The end products of the reactions described herein may be isolated by conventional techniques, e.g. by extraction, crystallisation, distillation, chromatography, etc.

### Biological Activity

The present invention is devoted to the provision novel modulators of the nicotinic receptors, which modulators are useful for the treatment of diseases or disorders related to the cholinergic receptors, and in particular the nicotinic acetylcholine receptor (nAChR). Preferred compounds of the invention show a pronounced nicotinic acetylcholine α7 receptor subtype selectivity.

Due to their pharmacological profile the compounds of the invention may be useful for the treatment of diseases or disorders as diverse as those related to the cholinergic system of the central nervous system (CNS), the peripheral nervous system (PNS), diseases or disorders related to smooth muscle contraction, endocrine diseases or disorders, diseases or disorders related to neuro-degeneration, diseases or disorders related to inflammation, pain, and withdrawal symptoms caused by the termination of abuse of chemical substances.

The compounds of the invention may also be useful as diagnostic tools or monitoring agents in various diagnostic methods, and in particular for in vivo receptor imaging (neuroimaging), and they may be used in labelled or unlabelled form.

In a preferred embodiment, the disease, disorder or condition contemplated according to the invention, and responsive to modulation of nicotinic acetylcholine receptors is anxiety, a cognitive disorder, a learning deficit, a memory deficit or dysfunction, Alzheimer's disease, attention deficit, attention deficit hyperactivity disorder, Parkinson's disease, Huntington's disease, Amyotrophic Lateral Sclerosis, Gilles de la Tourette's syndrome, depression, mania, manic depression, psychosis, schizophrenia, obsessive compulsive disorders (OCD), panic disorders, an eating disorder including anorexia nervosa, bulimia and obesity, narcolepsy, nociception, AIDS-dementia, senile dementia, peripheral neuropathy, autism, dyslexia, tardive dyskinesia, hyperkinesia, epilepsy, post-traumatic syndrome, social phobia, a sleeping disorder, pseudo dementia, Ganser's syndrome, pre-menstrual syndrome, late luteal phase syndrome, chronic fatigue syndrome, mutism, trichotillomania, jet-lag, hypertension, cardiac arrhythmias, a smooth muscle contraction disorder including convulsive disorders, angina pectoris, premature labour, convulsions, diarrhoea, asthma, epilepsy, tardive dyskinesia, hyperkinesia, premature ejaculation and erectile difficulty, an endocrine system disorder including thyrotoxicosis and pheochromocytoma, a neurodegenerative disorder, including transient anoxia and induced neuro-degeneration, pain, mild, moderate or severe pain, acute pain, chronic pain, pain of recurrent character, neuropathic pain, pain caused by migraine, postoperative pain, phantom limb pain, neuropathic pain, chronic headache, central pain, pain related to diabetic neuropathy, to postherpetic neuralgia or to peripheral nerve injury, an inflammatory disorder, including an inflammatory skin disorder, acne, rosacea, Crohn's disease, inflammatory bowel disease, ulcerative colitis and diarrhoea, a disorder associated with withdrawal symptoms caused by termination of use of addictive substances, including nicotine withdrawal symptoms, opioid withdrawal symptoms including heroin, cocaine and morphine, benzodiazepine withdrawal symptoms including benzodiazepine-like drugs and alcohol.

In a more preferred embodiment, the disease, disorder or condition responsive to modulation of nicotinic acetylcholine receptors is a cognitive disorder, psychosis, schizophrenia or depression.

In another more preferred embodiment, the disease, disorder or condition responsive to modulation of nicotinic acetylcholine receptors is associated with smooth muscle contractions, including convulsive disorders, angina pectoris, premature labour, convulsions, diarrhoea, asthma, epilepsy, tardive dyskinesia, hyperkinesia, premature ejaculation and erectile difficulty.

In still another more preferred embodiment, the disease, disorder or condition responsive to modulation of nicotinic acetylcholine receptors is related to the endocrine system, such as thyrotoxicosis and pheochromocytoma.

In yet another more preferred embodiment, the disease, disorder or condition responsive to modulation of nicotinic acetylcholine receptors is a neurodegenerative disorder including transient anoxia and induced neuro-degeneration.

In a further more preferred embodiment, the disease, disorder or condition responsive to modulation of nicotinic acetylcholine receptors is pain, including mild, moderate or even severe pain of acute, chronic or recurrent character, as well as pain caused by migraine, postoperative pain, and phantom limb pain. The pain may in particular be neuropathic pain, chronic headache, central pain, pain related to diabetic neuropathy, to postherpetic neuralgia, or to peripheral nerve injury.

In a further more preferred embodiment, the disease, disorder or condition responsive to modulation of nicotinic acetylcholine receptors is an inflammatory skin disorder such as acne and rosacea, Crohn's disease, inflammatory bowel disease, ulcerative colitis, and diarrhoea.

Finally the compounds of the invention may be useful for the treatment of withdrawal symptoms caused by termination of use of addictive substances. Such addictive substances include nicotine containing products such as tobacco, opioids such as heroin, cocaine and morphine, benzodiazepines and benzodiazepine-like drugs, and alcohol. Withdrawal from addictive substances is in general a traumatic experience characterised by anxiety and frustration, anger, anxiety, difficulties in concentrating, restlessness, decreased heart rate and increased appetite and weight gain.

In this context “treatment” covers treatment, prevention, prophylactics and alleviation of withdrawal symptoms and abstinence as well as treatment resulting in a voluntary diminished intake of the addictive substance.

### Pharmaceutical Compositions

In another aspect the invention provides novel pharmaceutical compositions comprising a therapeutically effective amount of 6-phenyl-pyrimidin-4-yl-(phenylamine or phenoxy) derivative of the invention.

While a 6-phenyl-pyrimidin-4-yl-(phenylamine or phenoxy) derivative of the invention for use in therapy may be administered in the form of the raw compound, it is preferred to introduce the active ingredient, optionally in the form of a physiologically acceptable salt, in a pharmaceutical composition together with one or more adjuvants, excipients, carriers, buffers, diluents, and/or other customary pharmaceutical auxiliaries.

In a preferred embodiment, the invention provides pharmaceutical compositions comprising the 6-phenyl-pyrimidin-4-yl-(phenylamine or phenoxy) derivative of the invention, or a pharmaceutically acceptable salt or derivative thereof, together with one or more pharmaceutically acceptable carriers therefore, and, optionally, other therapeutic and/or prophylactic ingredients know and used in the art. The carrier(s) must be “acceptable” in the sense of being compatible with the other ingredients of the formulation and not harmful to the recipient thereof.

The pharmaceutical composition of the invention may be administered by any convenient route, which suits the desired therapy. Preferred routes of administration include oral administration, in particular in tablet, in capsule, in drage, in powder, or in liquid form, and parenteral administration, in particular cutaneous, subcutaneous, intramuscular, or intravenous injection. The pharmaceutical composition of the invention can be manufactured by the skilled person by use of standard methods and conventional techniques appropriate to the desired formulation. When desired, compositions adapted to give sustained release of the active ingredient may be employed.

Further details on techniques for formulation and administration may be found in the latest edition of Remington's Pharmaceutical Sciences (Maack Publishing Co., Easton, Pa.).

The actual dosage depends on the nature and severity of the disease being treated, and is within the discretion of the physician, and may be varied by titration of the dosage to the particular circumstances of this invention to produce the desired therapeutic effect. However, it is presently contemplated that pharmaceutical compositions containing of from about 0.1 to about 500 mg of active ingredient per individual dose, preferably of from about 1 to about 100 mg, most preferred of from about 1 to about 10 mg, are suitable for therapeutic treatments.

The active ingredient may be administered in one or several doses per day. A satisfactory result can, in certain instances, be obtained at a dosage as low as 0.1 μg/kg i.v. and 1 μg/kg p.o. The upper limit of the dosage range is presently considered to be about 10 mg/kg i.v. and 100 mg/kg p.o. Preferred ranges are from about 0.1 μg/kg to about 10 mg/kg/day i.v., and from about 1 μg/kg to about 100 mg/kg/day p.o.

### Methods of Therapy

The 6-phenyl-pyrimidin-4-yl-(phenylamine or phenoxy) derivatives of the present invention are valuable nicotinic receptor modulators, and therefore useful for the treatment of a range of ailments involving cholinergic dysfunction as well as a range of disorders responsive to the action of nAChR modulators.

In another aspect the invention provides a method for the treatment, prevention or alleviation of a disease or a disorder or a condition of a living animal body, including a human, which disease, disorder or condition is responsive to modulation of cholinergic receptors, and which method comprises administering to such a living animal body, including a human, in need thereof an effective amount of a 6-phenyl-pyrimidin-4-yl-(phenylamine or phenoxy) derivative of the invention.

In the context of this invention the term “treatment” covers treatment, prevention, prophylaxis or alleviation, and the term “disease” covers illnesses, diseases, disorders and conditions related to the disease in question.

The preferred indications contemplated according to the invention are those stated above.

It is at present contemplated that suitable dosage ranges are 0.1 to 1000 milligrams daily, 10-500 milligrams daily, and especially 30-100 milligrams daily, dependent as usual upon the exact mode of administration, form in which administered, the indication toward which the administration is directed, the subject involved and the body weight of the subject involved, and further the preference and experience of the physician or veterinarian in charge.

A satisfactory result can, in certain instances, be obtained at a dosage as low as 0.005 mg/kg i.v. and 0.01 mg/kg p.o. The upper limit of the dosage range is about 10 mg/kg i.v. and 100 mg/kg p.o. Preferred ranges are from about 0.001 to about 1 mg/kg i.v. and from about 0.1 to about 10 mg/kg p.o.

### EXAMPLES

The invention is further illustrated with reference to the following examples, which are not intended to be in any way limiting to the scope of the invention as claimed.

### Example 1

### Preparatory Example

### General Synthetic Procedure for the Compounds of the Invention

Nucleophilic substitution of a commercial 4,6-dihalo-5-nitropyrimidine with the appropriate commercial aniline in boiling 2-propanol yielded the correspondent phenyl-pyrimidin-4-yl-amines (A), as previously described by Clark, R. L. et al. in Journal of Medicinal Chemistry 21(9), 965-965, 1978. Suzuki coupling reaction of these latter compounds with the suitably-substituted benzeneboronic acids afforded the (6-phenyl-5-nitro-pyrimidin-4-yl)-phenyl-amines (B), as suggested by Hauser D R J et al. in Journal of Medicinal Chemistry 2007 50 (9) 2060-2066. These latter were catalytically hydrogenated to give the correspondent (6-phenyl-5-amino-pyrim id in-4-yl)-phenyl-am ines (C). Those (6-phenyl-5-nitro-pyrimidin-4-yl)-phenyl-amines (B) and (6-phenyl-5-amino-pyrimidin-4-yl)-phenyl-amines (C) bearing a methoxy substituent (R3) were subjected to ether hydrolysis upon mild nucleophilic substitution with the Lewis acid boron tribromide, to afford the (6-phenyl-5-amino-pyrimidin-4-yl)-phenyl-amines (D).

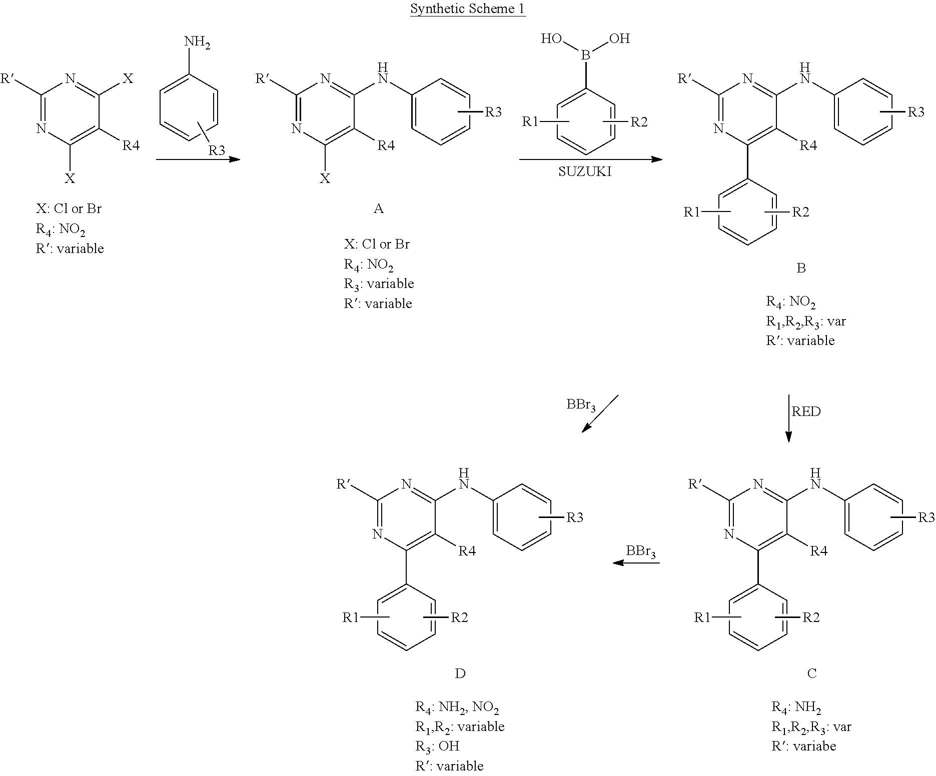

### Experimental Procedures

### Intermediates

### (6-Chloro-5-nitro-pvrimidin-4-yl)-(4-methoxy-phenyl)-amine (INT-1)

To a stirred and ice-cooled solution of 4,6-dichloro-5-nitropyrimidine (1.000 g, 5.1553 mmol, 1 eq) in anhydrous 2-propanol (10 ml), 4-methoxy-phenylamine (0.6984 g, 5.6708 mmol, 1.1 eq) and triethylamine (1.304 g, 12.888 mmol, 2.5 eq) were added drop-wise and the mixture refluxed under a nitrogen atmosphere for 2 hours. The resulting reaction mixture was evaporated, and the solid residue was suspended in water (100 ml) and extracted with chloroform (150 ml×3). The combined organic layers were washed with brine, dried over anhydrous sodium sulphate, filtered and evaporated, to afford the title compound (1.400 g, 96% mass balance) as a red solid. This latter was purified by column chromatography over silica gel eluting with 5% ethyl acetate in hexane (0.600 g, 41% yield; MH+ 281, 96% pure at LCMS).

### (6-Chloro-5-nitro-pyrimidin-4-yl)-(6-methoxy-pyridin-3-yl)-amine (INT-2)

To a stirred and ice-cooled solution of 4,6-dichloro-5-nitropyrimidine (2.000 g, 10.3105 mmol, 1 eq) in anhydrous 2-propanol (40 ml), 6-methoxy-pyridin-3-ylamine (1.408 g, 11.3416 mmol, 1.1 eq) and triethylamine (2.608 g, 25.7762 mmol, 2.5 eq) were added drop-wise and the mixture refluxed under a nitrogen atmosphere for 2 hours. The resulting reaction mixture was evaporated, and the solid residue was suspended in water (150 ml) and extracted with ethyl acetate (200 ml×3). The combined organic layers were washed with brine, dried over anhydrous sodium sulphate, filtered and evaporated, to afford the title compound (2.600 g, 96% mass balance) as a yellow solid. This latter was purified by column chromatography over silica gel eluting with 7% ethyl acetate in hexane (3.200 g, 74% yield; MH+ 282, 98% pure at LCMS).

### 4-(6-Chloro-5-nitro-pyrimidin-4-ylamino)-benzenesulfonamide (INT-3)

To a stirred and ice-cooled solution of 4,6-dichloro-5-nitropyrimidine (3.000 g, 15.4658 mmol, 1 eq) in anhydrous 2-propanol (30 ml), 4-amino-benzenesulfonamide (2.718 g, 15.4658 mmol, 1.1 eq) and triethylamine (3.130 g, 30.9316 mmol, 2 eq) were added drop-wise and the mixture refluxed under a nitrogen atmosphere for 3 hours. The resulting reaction mixture was evaporated, and the solid residue was suspended in water (150 ml) and extracted with ethyl acetate (200 ml×3). The combined organic layers were washed with brine, dried over anhydrous sodium sulphate, filtered and evaporated, to afford the title compound (4.500 g, 89% mass balance) as a yellow solid. This latter was purified by column chromatography over silica gel eluting with 15% ethyl acetate in hexane (1.400 g, 27% yield; MH+ 330, 93% pure at LCMS).

### (6-Chloro-5-nitro-pyrimidin-4-yl)-(1H-indol-5-yl)-amine (INT-4)

To a stirred and ice-cooled solution of 4,6-dichloro-5-nitropyrimidine (2.000 g, 10.3105 mmol, 1 eq) in anhydrous 2-propanol (20 ml), 1H-Indol-5-ylamine (1.363 g, 10.3105 mmol, 1 eq) and triethylamine (2.0866 g, 20.621 mmol, 2 eq) were added drop-wise and the mixture refluxed under a nitrogen atmosphere for 2 hours. The resulting reaction mixture was evaporated, and the solid residue was suspended in water (100 ml) and extracted with ethyl acetate (120 ml×3). The combined organic layers were washed with brine, dried over anhydrous sodium sulphate, filtered and evaporated, to afford the title compound (2.900 g, 97% mass balance) as a red solid. This latter was purified by column chromatography over silica gel eluting with 12% ethyl acetate in hexane (0.651 g, 21% yield; MH+ 289, 99% pure at LCMS).

### Compounds of the Invention

### [6-(2-Fluoro-4-trifluoromethyl-phenyl)-5-nitro-pyrimidin-4-yl]-(4-methoxy-phenyl)-amine (Compound 1.1)

A mixture of (6-chloro-5-nitro-pyrimidin-4-yl)-(4-methoxy-phenyl)-amine (INT-1; 0.500 g, 1.7815 mmol, 1 eq), 2-fluoro-4-(trifluoromethyl)phenylboronic acid (0.4075 g, 1.9597 mmol, 1.1 eq), sodium carbonate (0.3776 g, 3.563 mmol, 2 eq) and 1,4-dioxane (10 ml) was degassed with nitrogen and kept under a nitrogen atmosphere during the entire course of the reaction. To the degassed mixture, palladium (II) (bistriphenylphosphine)dichloride (0.0625 g, 0.0891 mmol, 0.05 eq) was added and the resulting reaction mixture, refluxed overnight and cooled to room temperature, was worked up by evaporation to dryness followed by addition of water and finally extracted with chloroform. The combined organic layers, dried over anhydrous MgSO4, afforded upon evaporation a red solid material (0.600 g), which eluted over silica gel (60-120 mesh) with 7% ethyl acetate in hexane gave 0.220 g (30% yield) of the pure title compound as an orange solid. M.p. 155.6-155.8° C. LC-ESI-HRMS of [M+H]+ shows 409.0904 Da. Calc. 409.092378 Da, dev. −4.8 ppm.

### 4-[6-(2-Fluoro-4-trifluoromethyl-phenyl)-5-nitro-pyrimidin-4-ylamino]-phenol (Compound 1.2)

To a solution of [6-(2-fluoro-4-trifluoromethyl-phenyl)-5-nitro-pyrimidin-4-yl]-(4-methoxy-phenyl)-amine (Compound 1; 0.400 g, 0.9796 mmol, 1 eq) in anhydrous dichloromethane (10 ml), cooled to −78° C. and kept under nitrogen, a solution of boron tribromide (0.9816 g, ˜0.37 ml, 5.7799 mmol, 4 eq) in anhydrous dichloromethane (5 ml) was added drop-wise. The reaction mixture was allowed to attain room temperature spontaneously and stirred overnight. The mixture was then cooled again in an ice-salt bath and the excess of the reagent was decomposed by treatment with methanol (10 ml) followed by water (15 ml) and finally extracted with chloroform. The combined organic layers, dried over anhydrous MgSO₄, afforded upon evaporation a black solid material (0.350 g), which eluted over silica gel (60-120 mesh) with 11% ethyl acetate in hexane gave 0.166 g (42% yield) of the pure (>99% at HPLC) title compound as a brown solid. M.p. 169-171.5° C. LC-ESI-HRMS of [M+H]+ shows 395.0754 Da. Calc. 395.076728 Da, dev. −3.4 ppm.

### 6-(2-Fluoro-4-trifluoromethyl-phenyl)-N*4*-(4-methoxy-Phenyl)-pyrimidine-4,5-diamine (Compound 1.3)

A degassed mixture of a solution of [6-(2-fluoro-4-trifluoromethyl-phenyl)-5-nitro-pyrimidin-4-yl]-(4-methoxy-phenyl)-amine (Compound 1; 1.000 g, 2.4491 mmol, 1 eq) in methanol (10 ml) and raney-nickel (0.100 g, ˜0.3 eq) was put under a hydrogen atmosphere and stirred at room temperature for 4 hours. The resulting reaction mixture was filtered through a celite bed, washed with methanol (50 ml×3) and the filtrate evaporated under reduced pressure to furnish a solid residue. This material was dissolved in chloroform and the organic layer, washed with water and dried over anhydrous MgSO₄, afforded upon evaporation 0.800 g (86% yield) of the title compound as a white solid, which is 99% pure at HPLC. M.p. 212,1-213.2° C. LC-ESI-HRMS of [M+H]+ shows 379.1172 Da. Calc. 379.118198 Da, dev. −2.6 ppm.

### 4-[5-Amino-6-(2-fluoro-4-trifluoromethyl-phenyl)-pyrimidin-4-ylamino]-phenol (Compound 1.4)

To a solution of 6-(2-fluoro-4-trifluoromethyl-phenyl)-N*4*-(4-methoxy-phenyl)-pyrimidine-4,5-diamine (Compound 3; 1.300 g, 3.4362 mmol, 1 eq) in anhydrous dichloromethane (15 ml), cooled to −78° C. and kept under nitrogen, a solution of boron tribromide (6.026 g, ˜2.3 ml, 24.0534 mmol, 7 eq) in anhydrous dichloromethane (10 ml) was added drop-wise. The reaction mixture was allowed to attain room temperature spontaneously and stirred overnight. The mixture was then cooled again in an ice-salt bath and the excess of the reagent was decomposed by treatment with methanol (20 ml) followed by water (25 ml) and finally extracted with chloroform. The combined organic layers, dried over anhydrous MgSO₄, afforded upon evaporation a white solid material (˜1.100 g), which eluted over silica gel (230-400 mesh) with 30% ethyl acetate in hexane gave 0.850 g (70% yield) of the pure (>99% at HPLC) title compound as an off-white solid. M.p. 194.2-195.7° C. LC-ESI-HRMS of [M+H]+ shows 365.1026 Da. Calc. 30 365.102548 Da, dev. 0.1 ppm.

### [6-(2-Fluoro-4-trifluoromethyl-phenyl)-5-nitro-pyrimidin-4-yl]-(6-methoxy-pyridin-3-yl)-amine (Compound 1.5)

To a degassed mixture of (6-chloro-5-nitro-pyrimidin-4-yl)-(6-methoxy-pyridin-3-yl)-amine (INT-2; 2.700 g, 9.5861 mmol, 1 eq), 2-fluoro-4-(trifluoromethyl)phenylboronic acid (2.192 g, 10.5447 mmol, 1.1 eq), sodium carbonate (2.540 g, 23.9652 mmol, 2.5 eq), 1,4-dioxane (40 ml) and water (20 ml), palladium (II) (bistriphenylphosphine)dichloride (0.3364 g, 0.4793 mmol, 0.05 eq) was added and the resulting reaction mixture, refluxed for 5 hours and cooled to room temperature, was worked up by concentration under reduced pressure followed by addition of water and finally extracted with chloroform. The combined organic layers, dried over anhydrous MgSO4, afforded upon evaporation a brown gummy material (3.200 g, 82% mass balance), which eluted over silica gel (60-120 mesh) with 7% ethyl acetate in hexane gave 1.700 g (36% yield) of the pure title compound as an orange solid. MH+ 410.

### 6-(2-Fluoro-4-trifluoromethyl-phenyl)-N*4*-(6-methoxy-rwrid in-3-yl)-pyrimidine-4,5-diamine (Compound 1.6)

A degassed mixture of a solution of [6-(2-Fluoro-4-trifluoromethyl-phenyl)-5-nitro-pyrimidin-4-yl]-(6-methoxy-pyridin-3-yl)-amine (Compound 4; 1.000 g, 2.4432 mmol, 1 eq) in methanol (30 ml) and raney-nickel (0.100 g, ˜0.3 eq) was put under a hydrogen atmosphere and stirred at room temperature overnight. The resulting reaction mixture was filtered through a celite bed, washed with methanol (50 ml×3) and the filtrate evaporated under reduced pressure to furnish a solid residue. This material was dissolved in chloroform and the organic layer, washed with water and dried over anhydrous MgSO₄, afforded upon evaporation 0.700 g (77% yield) of the title compound as a white solid, which is 96% pure at HPLC. M.p. 188.6-189.9° C.

### 4-[6-(2-Fluoro-4-trifluoromethyl-phenyl)-5-nitro-pyrimidin-4-ylamino]-benzenesulfonamide (Compound 1.7)

To a degassed mixture of 4-(6-chloro-5-nitro-pyrimidin-4-ylamino)-benzenesulfonamide (INT-3; 1.400 g, 4.246 mmol, 1 eq), 2-fluoro-4-(trifluoromethyl)phenylboronic acid (0.9711 g, 4.6706 mmol, 1.1 eq), sodium carbonate (1.125 g, 10.615 mmol, 2.5 eq), 1,4-dioxane (15 ml) and water (5 ml), palladium (II) (bistriphenylphosphine)dichloride (0.149 g, 0.2123 mmol, 0.05 eq) was added and the resulting reaction mixture, refluxed for 5 hours and cooled to room temperature, was worked up by concentration under reduced pressure followed by addition of water and finally extracted with chloroform. The combined organic layers, dried over anhydrous MgSO4, afforded upon evaporation a yellow solid material (1.700 g, 87% mass balance), which eluted over silica gel (230-400 mesh) with 12% ethyl acetate in hexane gave 0.503 g (32% yield) of the pure title compound as an orange solid. MH+ 458.

### 4-[5-Amino-6-(2-fluoro-4-trifluoromethyl-phenyl)-pyrimidin-4-ylamino]-benzenesulfonamide (Compound 1.8)

A degassed mixture of a solution of 4-[6-(2-Fluoro-4-trifluoromethyl-phenyl)-5-nitro-pyrimidin-4-ylamino]-benzenesulfonamide (Compound 6; 0.900 g, 1.9678 mmol, 1 eq) in methanol (15 ml) and raney-nickel (0.080 g, ˜0.3 eq) was put under a hydrogen atmosphere and stirred at room temperature overnight. The resulting reaction mixture was filtered through a celite bed, washed with methanol (50 ml×3) and the filtrate evaporated under reduced pressure to furnish a solid residue. This material was dissolved in chloroform and the organic layer, washed with water and dried over anhydrous MgSO₄, afforded upon evaporation 0.700 g (77% yield) of the title compound as a white solid. After washing with diethylether, the residual solid (0.402 g, 41% yield) resulted to be 96% pure at HPLC.

### [6-(2-Fluoro-4-trifluoromethyl-phenyl)-5-nitro-pyrimidin-4-yl]-(1H-indol-5-yl)-amine (Compound 1.9)

To a degassed mixture of (6-chloro-5-nitro-pyrimidin-4-yl)-(1H-indol-5-yl)-amine (INT-4; 1.200 g, 4.1425 mmol, 1 eq), 2-fluoro-4-(trifluoromethyl)phenylboronic acid (0.9474 g, 4.5568 mmol, 1.1 eq), sodium carbonate (1.0976 g, 10.3562 mmol, 2.5 eq), 1,4-dioxane (10 ml) and water (5 ml), palladium (II) (bistriphenylphosphine)dichloride (0.1454 g, 0.2071 mmol, 0.05 eq) was added and the resulting reaction mixture, refluxed for 5 hours and cooled to room temperature, was worked up by concentration under reduced pressure followed by addition of water and finally extracted with chloroform. The combined organic layers, dried over anhydrous MgSO4, afforded upon evaporation a yellow solid material (1.600 g, 94% mass balance), which eluted over silica gel (230-400 mesh) with 10% ethyl acetate in hexane gave 0.633 g (37% yield) of the pure title compound as a red solid. M.p. 147.4-148.9° C.

### 6-(2-Fluoro-4-trifluoromethyl-phenyl)-N*4*-(1H-indol-5-yl)-pyrimidine-4,5-diamine (Compound 1.10)

A degassed mixture of a solution of [6-(2-fluoro-4-trifluoromethyl-phenyl)-5-nitro-pyrimidin-4-yl]-(1H-indol-5-yl)-amine (Compound 8; 0.500 g, 1.1981 mmol, 1 eq) in methanol (15 ml) and raney-nickel (0.049 g, ˜0.3 eq) was put under a hydrogen atmosphere and stirred at room temperature overnight. The resulting reaction mixture was filtered through a celite bed, washed with methanol (50 ml×3) and the filtrate evaporated under reduced pressure to furnish a solid residue. This material was dissolved in chloroform and the organic layer, washed with water and dried over anhydrous MgSO₄, afforded upon evaporation 0.450 g (97% yield) of the title compound as a white solid. After washing with chloroform, the residual solid (0.241 g, 51% yield) resulted to be 96% pure at HPLC. MH+ 387.

In analogy with the procedure described above the following compounds were made.

### 5-[5-Amino-6-(2-fluoro-4-trifluoromethyl-phenyl)-pyrimidin-4-ylamino]-pyridin-2-ol (Compound 1.11)

LC-ESI-HRMS of [M+H]+ shows 366.0957454 Da. Calc. 366.097252 Da, dev. −4.1 ppm.

### 6-(2-Fluoro-4-trifluoromethyl-phenyl)-N*4*-(1H-indazol-5-yl)-pyrimidine-4,5-diamine (Compound 1.12)

LC-ESI-HRMS of [M+H]+ shows 389.1131 Da. Calc. 389.113236 Da, dev. −0.3 ppm.

### N*4*-Benzo[1,3]dioxol-5-yl-6-(2-fluoro-4-trifluoromethyl-phenyl)-pyrimidine-4,5-diamine (Compound 1.13)

LC-ESI-HRMS of [M+H]+ shows 393.098728111807 Da. Calc. 393.096918 Da, dev. 4.6 ppm.

### N*4*-(4-Cyclopropylmethoxy-phenyl)-6-(2-fluoro-4-trifluoromethyl-phenyl)-pyrimidine-4,5-diamine (Compound 1.14)

LC-ESI-HRMS of [M+H]+ shows 419.1494 Da. Calc. 419.148953 Da, dev. 1.1 ppm.

### 6-(2-Fluoro-4-trifluoromethyl-phenyl)-2-methyl-N*4*-naphthalen-1-yl-pyrimidine-4,5-diamine (Compound 1.15)

LC-ESI-HRMS of [M+H]+ shows 413.13845 Da. Calc. 413.138388 Da, dev. 0.2 ppm.

### 6-(2-Fluoro-4-trifluoromethyl-phenyl)-N*4*-(4-methoxy-phenyl)-2-methyl-pyrimidine-4,5-diamine (Compound 1.16)

LC-ESI-HRMS of [M+H]+ shows 393.13371 Da. Calc. 393.133303 Da, dev. 1 ppm.

### N*4*-(2,4-Difluoro-phenyl)-6-(2-fluoro-4-trifluoromethyl-phenyl)-2-methyl-pyrimidine-4,5-diamine (Compound 1.17)

LC-ESI-HRMS of [M+H]+ shows 399.10395 Da. Calc. 399.103894 Da, dev. 0.1 ppm.

### N*4*-(4-Fluoro-phenyl)-6-(2-fluoro-4-trifluoromethyl-phenyl)-2-methyl-pyrimidine-4,5-diamine (Compound 1.18)

LC-ESI-HRMS of [M+H]+ shows 381.11384 Da. Calc. 381.113316 Da, dev. 1.4 ppm.

### N*4*-(2-Fluoro-phenyl)-6-(2-fluoro-4-trifluoromethyl-phenyl)-2-methyl-pyrimidine-4,5-diamine (Compound 1.19)

LC-ESI-HRMS of [M+H]+ shows 381.11388 Da. Calc. 381.113316 Da, dev. 1.5 ppm.

### N*4*-(2,4-Dichloro-phenyl)-6-(2-fluoro-4-trifluoromethyl-phenyl)-2-methyl-pyrimidine-4,5-diamine (Compound 1.20)

LC-ESI-HRMS of [M+H]+ shows 431.04512 Da. Calc. 431.044794 Da, dev. 0.8 ppm.

### N*4*-Benzo[1,3]dioxol-5-yl-6-(2-fluoro-4-trifluoromethyl-phenyl)-2-methyl-pyrimidine-4,5-diamine (Compound 1.21)

LC-ESI-HRMS of [M+H]+ shows 407.11266 Da. Calc. 407.112568 Da, dev. 0.2 ppm.

### N*4*-(3,5-Dimethoxy-phenyl)-6-(2-fluoro-4-trifluoromethyl-phenyl)-2-methyl-pyrimidine-4,5-diamine (Compound 1.22)

LC-ESI-HRMS of [M+H]+ shows 423.14385 Da. Calc. 423.143868 Da, dev. 0 ppm.

### N*4*-(4-Chloro-3-trifluoromethyl-phenyl)-6-(2-fluoro-4-trifluoromethyl-phenyl)-2-methyl-pyrimidine-4,5-diamine (Compound 1.23)

LC-ESI-HRMS of [M+H]+ shows 465.07081 Da. Calc. 465.07115 Da, dev. −0.7 ppm.

### N*4*-(3-Fluoro-phenyl)-6-(2-fluoro-4-trifluoromethyl-phenyl)-2-methyl-pyrimidine-4,5-diamine (Compound 1.24)

LC-ESI-HRMS of [M+H]+ shows 381.11375 Da. Calc. 381.113316 Da, dev. 1.1 ppm.

### N*4*-(4-Cyclohexyl-phenyl)-6-(2-fluoro-4-trifluoromethyl-phenyl)-2-methyl-pyrimidine-4,5-diamine (Compound 1.25)

LC-ESI-HRMS of [M+H]+ shows 445.20112 Da. Calc. 445.200988 Da, dev. 0.3 ppm.

### N*4*-(4-Ethoxy-phenyl)-6-(2-fluoro-4-trifluoromethyl-phenyl)-2-methyl-pyrimidine-4,5-diamine (Compound 1.26)

LC-ESI-HRMS of [M+H]+ shows 407.14925 Da. Calc. 407.148953 Da, dev. 0.7 ppm.

### 6-(2-Fluoro-4-trifluoromethyl-phenyl)-2-methyl-N*4*-p-tolyl-pyrimidine-4,5-diamine (Compound 1.27)

LC-ESI-HRMS of [M+H]+ shows 377.13879 Da. Calc. 377.138388 Da, dev. 1.1 ppm.

### 6-(2-Fluoro-4-trifluoromethyl-phenyl)-N*4*-isoquinolin-5-yl-2-methyl-pyrimidine-4,5-diamine (Compound 1.28)

### N*4*-(2,3-Dihydro-benzo[1,4 dioxin-6-yl)-6-(2-fluoro-4-trifluoromethyl-phenyl)-2-methyl-pyrimidine-4,5-diamine (Compound 1.29)

LC-ESI-HRMS of [M+H]+ shows 421.12824 Da. Calc. 421.128218 Da, dev. 0 ppm.

### N*4*-(3,4-Dimethoxy-phenyl)-6-(2-fluoro-4-trifluoromethyl-phenyl)-2-methyl-pyrimidine-4,5-diamine (Compound 1.30)

LC-ESI-HRMS of [M+H]+ shows 423.14401 Da. Calc. 423.143868 Da, dev. 0.3 ppm.

### 6-(2-Fluoro-4-trifluoromethyl-phenyl)-N*4*-(3-methoxy-phenyl)-2-methyl-pyrimidine-4,5-diamine (Compound 1.31)

LC-ESI-HRMS of [M+H]+ shows 393.13367 Da. Calc. 393.133303 Da, dev. 0.9 ppm.

### 6-(2-Fluoro-4-trifluoromethyl-phenyl)-2-methyl-N*4*-(3-methylsulfanyl-phenyl)-pyrimidine-4,5-diamine (Compound 1.32)

LC-ESI-HRMS of [M+H]+ shows 409.11069 Da. Calc. 409.110459 Da, dev. 0.6 ppm.

### N*4*-(2,4-Dimethoxy-phenyl)-6-(2-fluoro-4-trifluoromethyl-phenyl)-2-methyl-pyrimidine-4,5-diamine (Compound 1.33)

LC-ESI-HRMS of [M+H]+ shows 423.14369 Da. Calc. 423.143868 Da, dev. −0.4 ppm.

### 6-(2-Fluoro-4-trifluoromethyl-phenyl)-2-methyl-N*4*-naphthalen-2-v1-pyrimidine-4,5-diamine (Compound 1.34)

LC-ESI-HRMS of [M+H]+ shows 413.13855 Da. Calc. 413.138388 Da, dev. 0.4 ppm.

### Example 2

### Preparatory Example

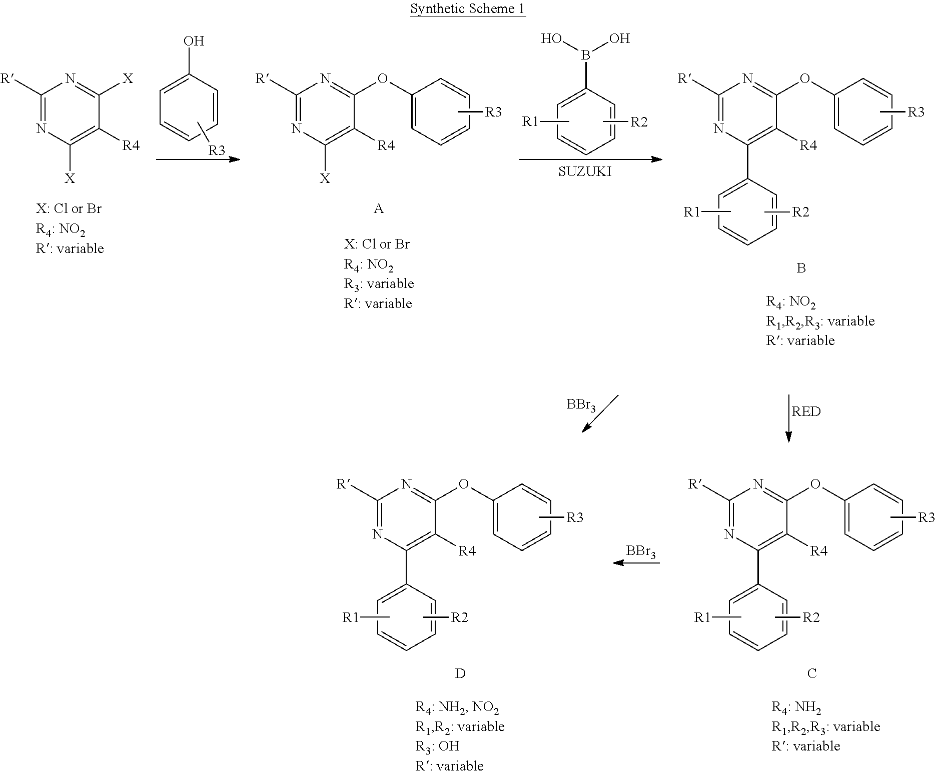

### 4-Chloro-6-(4-methoxy-phenoxy)-5-nitro-pyrimidine (Intermediate compound A)

To a stirred and ice-cooled solution of 4,6-dichloro-5-nitropyrimidine (3.000 g, 15.4658 mmol, 1 eq) in anhydrous 2-propanol (30 ml), 4-methoxyphenol (2.112 g, 17.0124 mmol, 1.1 eq) and triethylamine (3.913 g, 38.6645 mmol, 2.5 eq) were added drop-wise and the mixture stirred at room temperature under a nitrogen atmosphere for 2 hours. The resulting reaction mixture was evaporated, and the solid residue was suspended in water and extracted with ethyl acetate. The combined organic layers were washed with brine, dried over anhydrous sodium sulphate, filtered and evaporated, to afford the title compound (4.120 g, 100% mass balance) as a yellow solid. This latter was purified by column chromatography over silica gel eluting with 6-10% ethyl acetate in petroleum ether (2.801g, 61% yield; MH+ 282, >97% pure at LCMS).

### 4-(2-Fluoro-4-trifluoromethyl-phenyl)-6-(4-methoxy-phenoxy)-5-nitro-pyrimidine (Compound 2.1)

A mixture of 4-chloro-6-(4-methoxy-phenoxy)-5-nitro-pyrimidine (Compound A; 2.300 g, 8.166 mmol, 1 eq), 2-fluoro-4-(trifluoromethyl)phenylboronic acid (1.868 g, 8.9826 mmol, 1.1 eq), sodium carbonate (1.731 g, 16.332 mmol, 2 eq), 1,2 dimethoxyethane (15 ml) and water (7 ml) was degassed with nitrogen and kept under a nitrogen atmosphere during the entire course of the reaction. To the degassed mixture, palladium (II) (bistriphenylphosphine)dichloride (0.287 g, 0.4083 mmol, 0.05 eq) was added and the resulting reaction mixture, refluxed for 2 hours and cooled to room temperature, was worked up by evaporation to dryness followed by addition of water and finally extracted with ethyl acetate. The combined organic layers, dried over anhydrous MgSO4, afforded upon evaporation a brown gummy material (˜3.35 g), which eluted over silica gel (230-400 mesh) with 10% ethyl acetate in petroleum ether gave 0.965 g (˜30% yield) of the pure title compound as an off-white solid.

M.p. 203.8° C.-205.2° C. LC-ESI-HRMS of [M+H]+ shows 410.0764 Da. Calc. 410.075849 Da, dev. 1.3 ppm.

### 4-(2-Fluoro-4-trifluoromethyl-phenyl)-6-(4-methoxy-phenoxy)-pyrimidin-5-ylamine (Compound 2.2)

A degassed mixture of a solution of 4-(2-fluoro-4-trifluoromethyl-phenyl)-6-(4-methoxy-phenoxy)-5-nitro-pyrimidine (Compound 11; 1.500 g, 3.6648 mmol, 1 eq) in methanol (50 ml) and raney-nickel (0.150 g, ˜0.3 eq) was put under a hydrogen atmosphere and stirred at room temperature for 3 hours. The resulting reaction mixture was filtered through a celite bed, washed with methanol and the filtrate evaporated under reduced pressure to furnish a solid residue (˜1.35 g). This latter material was purified by elution over neutral alumina with 10% ethyl acetate in petroleum, to obtain 0.850 g (61% yield) of the title compound as a white solid.

M.p. 129.0°C.-130.4° C. LC-ESI-HRMS of [M+H]+ shows 380.1027 Da. Calc. 380.101669 Da, dev. 2.7 ppm.

### 4-[5-Amino-6-(2-fluoro-4-trifluoromethyl-phenyl)-pyrimidin-4-yloxyl-phenol (Compound 2.3)

To a solution of [4-(2-fluoro-4-trifluoromethyl-phenyl)-6-(4-methoxy-phenoxy)-pyrimidin-5-ylamine (Compound 12; 0.500 g, 1.3182 mmol, 1 eq) in anhydrous dichloromethane (20 ml), cooled to -78° C. and kept under nitrogen, a solution of boron tribromide (2.312 g, ˜0.87 ml, 9.2274 mmol, 7 eq) in anhydrous dichloromethane (5 ml) was added drop-wise. The reaction mixture was allowed to attain room temperature spontaneously and stirred overnight. The mixture was then cooled again in an ice-salt bath and the excess of the reagent was decomposed by treatment with methanol (10 ml) followed by water (15 ml) and finally extracted with chloroform. The combined organic layers, dried over anhydrous MgSO₄, afforded upon evaporation a yellow solid material (˜0.45 g), which eluted over neutral alumina with 25% ethyl acetate in petroleum ether gave 0.330 g (68% yield) of the pure title compound as a white solid.

M.p. 212.2° C.-213.5° C. LC-ESI-HRMS of [M+H]+ shows 366.0855 Da. Calc. 366.086019 Da, dev. −1.4 ppm.

In analogy with the procedure described above the following compounds were made.

### 4-(2-Fluoro-4-trifluoromethyl-phenyl)-2-methyl-6-(2-propyl-phenoxy)-Pyrimidin-5-ylamine (Compound 2.4)

LC-ESI-HRMS of [M+H]+ shows 406.15429 Da. Calc. 406.153704 Da, dev. 1.4 ppm.

### 4-(2-Fluoro-4-trifluoromethyl-phenyl)-2-methyl-6-(2-morpholin-4-yl-phenoxy)-pyrimidin-5-ylamine (Compound 2.5)

LC-ESI-HRMS of [M+H]+ shows 449.1593 Da. Calc. 449.159518 Da, dev. −0.5 ppm.

### 4-(2-Fluoro-4-trifluoromethyl-phenyl)-2-methyl-6-(quinolin-8-yloxy)-Pyrimidin-5-ylamine (Compound 2.6)

LC-ESI-HRMS of [M+H]+ shows 415.11798 Da. Calc. 415.117653 Da, dev. 0.8 ppm.

### 4-(2-Fluoro-4-trifluoromethyl-phenyl)-2-methyl-6-(5,6,7,8-tetrahydro-naphthalen-2-yloxy)-Pyrimidin-5-ylamine (Compound 2.7)

LC-ESI-HRMS of [M+H]+ shows 418.15389 Da. Calc. 418.153704 Da, dev. 0.4 ppm.

### 4-(4-Fluoro-phenoxy)-6-(2-fluoro-4-trifluoromethyl-phenyl)-2-methyl-pyrimidin-5-ylamine (Compound 2.8)

LC-ESI-HRMS of [M+H]+ shows 382.09756 Da. Calc. 382.097332 Da, dev. 0.6 ppm.

### 4-(Benzo[1,3 dioxol-5-yloxy)-6-(2-fluoro-4-trifluoromethyl-phenyl)-2-methyl-pyrimidin-5-ylamine (Compound 2.9)

LC-ESI-HRMS of [M+H]+ shows 408.09633 Da. Calc. 408.096584 Da, dev. −0.6 ppm.

### 4-(2-Chloro-4-methoxy-phenoxy)-6-(2-fluoro-4-trifluoromethyl-phenyl)-2-methyl-pyrimidin-5-ylamine (Compound 2.10)

LC-ESI-HRMS of [M+H]+ shows 428.07825 Da. Calc. 428.078347 Da, dev. −0.2 ppm.

### 4-(3-Fluoro-phenoxy)-6-(2-fluoro-4-trifluoromethyl-phenyl)-2-methyl-pyrimidin-5-ylamine (Compound 2.11)

LC-ESI-HRMS of [M+H]+ shows 382.09779 Da. Calc. 382.097332 Da, dev. 1.2 ppm.

### 4-(2-Chloro-4-trifluoromethyl-phenoxy)-6-(2-fluoro-4-trifluoromethyl-phenyl)-2-methyl-pyrimidin-5-ylamine (Compound 2.12)

LC-ESI-HRMS of [M+H]+ shows 466.05462 Da. Calc. 466.055166 Da, dev. −1.2 ppm.

### 4-(2-Fluoro-4-trifluoromethyl-phenyl)-6-(4-methoxy-phenoxy)-2-methyl-pyrimidin-5-ylamine (Compound 2.13)

LC-ESI-HRMS of [M+H]+ shows 394.11735 Da. Calc. 394.117319 Da, dev. 0.1 ppm.

### 4-(3-Chloro-5-methoxy-phenoxy)-6-(2-fluoro-4-trifluoromethyl-phenyl)-2-methyl-pyrimidin-5-ylamine (Compound 2.14)

LC-ESI-HRMS of [M+H]+ shows 428.0787 Da. Calc. 428.078347 Da, dev. 0.8 ppm.

### 4-(3-tert-Butyl-phenoxy)-6-(2-fluoro-4-trifl uoromethyl-phenyl)-2-methyl-pyrim id in-5-ylamine (Compound 2.15)

LC-ESI-HRMS of [M+H]+ shows 420.16921 Da. Calc. 420.169354 Da, dev. −0.4 ppm.

### 4-(4-tert-Butyl-phenoxy)-6-(2-fl uoro-4-trifluoromethyl-phenyl)-2-methyl-pyrimidin-5-ylamine (Compound 2.16)

LC-ESI-HRMS of [M+H]+ shows 420.16943 Da. Calc. 420.169354 Da, dev. 0.2 ppm.

### 4-(2-Fluoro-4-trifluoromethyl-phenyl)-2-methyl-6-(2-piperidin-1-yl-phenoxy)-pyrimidin-5-ylamine (Compound 2.17)

LC-ESI-HRMS of [M+H]+ shows 447.18022 Da. Calc. 447.180253 Da, dev. −0.1 ppm.

### 4-(2-Fluoro-phenoxy)-6-(2-fluoro-4-trifluoromethyl-phenyl)-2-methyl-pyrimidin-5-ylamine (Compound 2.18)

LC-ESI-HRMS of [M+H]+ shows 382.09758 Da. Calc. 382.097332 Da, dev. 0.6 ppm.

### 4-(2-Fluoro-4-trifluoromethyl-phenyl)-2-methyl-6-phenoxy-pyrimidin-5-ylamine (Compound 2.19)

LC-ESI-HRMS of [M+H]+ shows 364.10723 Da. Calc. 364.106754 Da, dev. 1.3 ppm.

### Example 3

### Biological Activity

In this example the positive modulation of wild-type nAChR α7 receptors by the compounds of the invention was determined using nAChR α7 receptors heterologously expressed in Xenopus laevis oocytes.

The electrical current through the nAChR α7 channel was measured using conventional two-electrode voltage clamp and nAChR α7 currents were activated by applying pulses of agonist-containing solution onto the nAChR α7 expressing oocyte.

In brief, the oocytes were placed in a recording chambers and continuously super-fused with an Oocyte Ringer (OR) solution containing 90 mM NaCl, 2.5 mM KCl, 2.5 mM CaCl₂, 1 mM MgCl₂ and 5 mM HEPES (pH adjusted to 7.4). The oocytes were clamped at −60 mV and currents were induced by applying 20 s pulses of 100 μM acetylcholine dissolved in OR. The intervals between the acetylcholine applications were 5 minutes, during which the oocytes were washed with OR. The first three applications were control applications to insure a constant response level of 100 μM acetylcholine. For the subsequent test applications, increasing concentrations (0.01-31.6 μM) of the test compound were applied 30 s before and during the acetylcholine (100 μM) application, which caused a robust increase in the acetylcholine-induced current amplitude.

The positive modulation in the presence of Compound was calculated as (test-control)/control×100% and the concentration response curve for this positive modulation was fitted to the sigmoidal logistic equation:

I=Iₘₐₓ/(1+(EC₅₀/[compound])ⁿ),

where Iₘₐₓ represents the maximal modulation of the control response, EC₅₀ is the concentration causing a half maximal response, and n is the slope coefficient.

Calculated EC₅₀ and Iₘₐₓ values are presented in the table below.

This is an indication of a biological activity as potent modulators of the nicotinic acetylcholine oc7 receptor subtype.

| Compound | EC₅₀ (μM) | Iₘₐₓ (%) |
|---|---|---|
| 1.1 | | 2 |
| 1.2 | 23 | 391 |
| 1.3 | 2.3 | 1123 |
| 1.5 | 6.6 | 137 |
| 1.6 | | 129 |
| 1.8 | 6.8 | 237 |
| 1.9 | 6.9 | 527 |
| 1.10 | 0.24 | 631 |
| 1.11 | | 22 |
| 1.12 | 9.9 | 184 |
| 1.13 | 54 | 848 |
| 1.14 | | 3 |
| 1.16 | 4.9 | 495 |
| 1.27 | 3.4 | 503 |
| 1.29 | 9.4 | 297 |
| 1.31 | 5.7 | 193 |
| 1.32 | 9.8 | 245 |
| 1.33 | 1.1 | 11 |
| 1.34 | 4.3 | 148 |
| 2.1 | | 19 |
| 2.2 | 6.6 | 119 |
| 2.3 | 15 | 104 |
| 2.9 | 0.74 | 35 |
| 2.13 | 6 | 42 |

1. A 6-phenyl-pyrimidin-4-yl-(phenylamine or phenoxy) derivative represented by Formula I 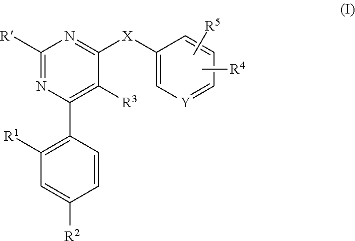
a stereoisomer thereof or a mixture of its stereoisomers, or a pharmaceutically acceptable salt thereof, wherein
X represents O or NH;
Y represents CH or N;
R′ represents hydrogen or alkyl;
R¹ and R², independently of each other, represent a substituent selected from the group consisting of hydrogen, halo, trifluoromethyl, trifluoromethoxy, nitro, cyano and hydroxy;
R³ represents amino or nitro; and
R⁴ and R⁵, independently of each other, represent hydrogen, halo, trifluoromethyl, hydroxy, alkoxy, thioalkoxy, cycloalkoxy, alkyl, cycloalkyl, sulfamoyl, piperidinyl, morpholinyl, or pyridinyl; or
R⁴ and R⁵ together with the phenyl ring to which they are attached form a methylenedioxy group or ethylenedioxy group; or
R⁴ and R⁵ together with the phenyl ring to which they are attached form a bicyclic carbocyclic ring selected from naphthyl and tetrahydronaphthalenyl; or
R⁴ and R⁵ together with the phenyl ring to which they are attached form a bicyclic heterocyclic ring selected from indolyl, indazolyl, quinolinyl and isoquinolinyl.

2. The 6-phenyl-pyrimidin-4-yl-(phenylamine or phenoxy) derivative of claim 1, a stereoisomer thereof or a mixture of its stereoisomers, or a pharmaceutically acceptable salt thereof, wherein X represents O or NH.

3. The 6-phenyl-pyrimidin-4-yl-(phenylamine or phenoxy) derivative of claim 1, a stereoisomer thereof or a mixture of its stereoisomers, or a pharmaceutically acceptable salt thereof, wherein Y represents CH or N.

4. The 6-phenyl-pyrimidin-4-yl-(phenylamine or phenoxy) derivative of claim 1, a stereoisomer thereof or a mixture of its stereoisomers, or a pharmaceutically acceptable salt thereof, wherein R′ represents hydrogen or alkyl.

5. The 6-phenyl-pyrimidin-4-yl-(phenylamine or phenoxy) derivative of claim 1, a stereoisomer thereof or a mixture of its stereoisomers, or a pharmaceutically acceptable salt thereof, wherein R¹ and R², independently of each other, represent a substituent selected from the group consisting of hydrogen, halo, trifluoromethyl, trifluoromethoxy, nitro, cyano and hydroxy.

6. The 6-phenyl-pyrimidin-4-yl-(phenylamine or phenoxy) derivative of claim 1, a stereoisomer thereof or a mixture of its stereoisomers, or a pharmaceutically acceptable salt thereof, wherein R³ represents amino or nitro.

7. The 6-phenyl-pyrimidin-4-yl-(phenylamine or phenoxy) derivative of claim 1, a stereoisomer thereof or a mixture of its stereoisomers, or a pharmaceutically acceptable salt thereof, wherein R⁴ and R⁵, independently of each other, represent hydrogen, halo, trifluoromethyl, hydroxy, alkoxy, thioalkoxy, cycloalkoxy, alkyl, cycloalkyl, sulfamoyl, piperidinyl, morpholinyl, or pyridinyl.

8. The 6-phenyl-pyrimidin-4-yl-(phenylamine or phenoxy) derivative of claim 1, a stereoisomer thereof or a mixture of its stereoisomers, or a pharmaceutically acceptable salt thereof, wherein R⁴ and R⁵ together with the phenyl ring to which they are attached form a methylenedioxy group or ethylenedioxy group.

9. The 6-phenyl-pyrimidin-4-yl-(phenylamine or phenoxy) derivative of claim 1, a stereoisomer thereof or a mixture of its stereoisomers, or a pharmaceutically acceptable salt thereof, wherein R⁴ and R⁵ together with the phenyl ring to which they are attached form a bicyclic carbocyclic ring selected from naphthyl and tetrahydronaphthalenyl.

10. The 6-phenyl-pyrimidin-4-yl-(phenylamine or phenoxy) derivative of claim 1, a stereoisomer thereof or a mixture of its stereoisomers, or a pharmaceutically acceptable salt thereof, wherein R⁴ and R⁵ together with the phenyl ring to which they are attached form a bicyclic heterocyclic ring selected from indolyl, indazolyl, quinolinyl and isoquinolinyl.

11. The 6-phenyl-pyrimidin-4-yl-(phenylamine or phenoxy) derivative of claim 1, which is
[6-(2-Fluoro-4-trifluoromethyl-phenyl)-5-nitro-pyrimidin-4-yl]-(4-methoxy-phenyl)-amine;
4-[6-(2-Fluoro-4-trifluoromethyl-phenyl)-5-nitro-pyrimidin-4-ylamino]-phenol;
6-(2-Fluoro-4-trifluoromethyl-phenyl)-N*4*-(4-methoxy-phenyl)-pyrimidine-4,5-diamine;
4-[5-Amino-6-(2-fluoro-4-trifluoromethyl-phenyl)-pyrimidin-4-ylamino]-phenol;
[6-(2-Fluoro-4-trifluoromethyl-phenyl)-5-nitro-pyrimidin-4-yl]-(6-methoxy-pyridin-3-yl)-amine;
6-(2-Fluoro-4-trifluoromethyl-phenyl)-N*4*-(6-methoxy-pyridin-3-yl)-pyrimidine-4,5-diamine;
4-[6-(2-Fluoro-4-trifluoromethyl-phenyl)-5-nitro-pyrimidin-4-ylamino]-benzenesulfonamide;
4-[5-Amino-6-(2-fluoro-4-trifluoromethyl-phenyl)-pyrimidin-4-ylamino]-benzenesulfonamide;
[6-(2-Fluoro-4-trifluoromethyl-phenyl)-5-nitro-pyrimidin-4-yl]-(1H-indol-5-yl)-amine;
6-(2-Fluoro-4-trifluoromethyl-phenyl)-N*4*-(1H-indol-5-yl)-pyrimidine-4,5-diamine;
5-[5-Amino-6-(2-fluoro-4-trifluoromethyl-phenyl)-pyrimidin-4-ylamino]-pyridin-2-ol;
6-(2-Fluoro-4-trifluoromethyl-phenyl)-N*4*-(1H-indazol-5-yl)-pyrimidine-4,5-diamine;
N*4*-Benzo[1,3]dioxol-5-yl-6-(2-fluoro-4-trifluoromethyl-phenyl)-pyrimidine-4,5-diamine;
N*4*-(4-Cyclopropylmethoxy-phenyl)-6-(2-fluoro-4-trifluoromethyl-phenyl)-pyrimidine-4,5-diamine;
6-(2-Fluoro-4-trifluoromethyl-phenyl)-2-methyl-N*4*-naphthalen-1-yl-pyrimidine-4,5-diamine;
6-(2-Fluoro-4-trifluoromethyl-phenyl)-N*4*-(4-methoxy-phenyl)-2-methyl-pyrimidine-4,5-diamine;
N*4*-(2,4-Difluoro-phenyl)-6-(2-fluoro-4-trifluoromethyl-phenyl)-2-methyl-pyrimidine-4,5-diamine
N*4*-(4-Fluoro-phenyl)-6-(2-fluoro-4-trifluoromethyl-phenyl)-2-methyl-pyrimidine-4,5-diamine;
N*4*-(2-Fluoro-phenyl)-6-(2-fluoro-4-trifluoromethyl-phenyl)-2-methyl-pyrimidine-4,5-diamine;
N*4*-(2,4-Dichloro-phenyl)-6-(2-fluoro-4-trifluoromethyl-phenyl)-2-methyl-pyrimidine-4,5-diamine;
N*4*-Benzo[1,3]dioxol-5-yl-6-(2-fluoro-4-trifluoromethyl-phenyl)-2-methyl-pyrimidine-4,5-diamine;
N*4*-(3,5-Dimethoxy-phenyl)-6-(2-fluoro-4-trifluoromethyl-phenyl)-2-methyl-pyrimidine-4,5-diamine;
N*4*-(4-Chloro-3-trifluoromethyl-phenyl)-6-(2-fluoro-4-trifluoromethyl-phenyl)-2-methyl-pyrimidine-4,5-diamine;
N*4*-(3-Fluoro-phenyl)-6-(2-fluoro-4-trifluoromethyl-phenyl)-2-methyl-pyrimidine-4,5-diamine;
N*4*-(4-Cyclohexyl-phenyl)-6-(2-fluoro-4-trifluoromethyl-phenyl)-2-methyl-pyrimidine-4,5-diamine;
N*4*-(4-Ethoxy-phenyl)-6-(2-fluoro-4-bifluoromethyl-phenyl)-2-methyl-pyrimidine-4,5-diamine;
6-(2-Fluoro-4-trifluoromethyl-phenyl)-2-methyl-N*4*-p-tolyl-pyrimidine-4,5-diamine;
6-(2-Fluoro-4-trifluoromethyl-phenyl)-N*4*-isoquinolin-5-yl-2-methyl-pyrimidine-4,5-diamine;
N*4*-(2,3-Dihydro-benzo[1,4]dioxin-6-yl)-6-(2-fluoro-4-trifluoromethyl-phenyl)-2-methyl-pyrimidine-4,5-diamine;
N*4*-(3,4-Dimethoxy-phenyl)-6-(2-fluoro-4-trifluoromethyl-phenyl)-2-methyl-pyrimidine-4,5-diamine;
6-(2-Fluoro-4-trifluoromethyl-phenyl)-N*4*-(3-methoxy-phenyl)-2-methyl-pyrimidine-4,5-diamine;
6-(2-Fluoro-4-trifluoromethyl-phenyl)-2-methyl-N*4*-(3-methylsulfanyl-phenyl)-pyrimidine-4,5-diamine;
N*4*-(2,4-Dimethoxy-phenyl)-6-(2-fluoro-4-trifluoromethyl-phenyl)-2-methyl-pyrimidine-4,5-diamine;
6-(2-Fluoro-4-trifluoromethyl-phenyl)-2-methyl-N*4*-naphthalen-2-yl-pyrimidine-4,5-diamine;
4-(2-Fluoro-4-trifluoromethyl-phenyl)-6-(4-methoxy-phenoxy)-5-nitro-pyrimidine;
4-(2-Fluoro-4-trifluoromethyl-phenyl)-6-(4-methoxy-phenoxy)-pyrimidin-5-ylamine;
4-[5-Amino-6-(2-fluoro-4-trifluoromethyl-phenyl)-pyrimidin-4-yloxy]-phenol;
4-(2-Fluoro-4-trifluoromethyl-phenyl)-2-methyl-6-(2-propyl-phenoxy)-pyrimidin-5-ylamine;
4-(2-Fluoro-4-trifluoromethyl-phenyl)-2-methyl-6-(2-morpholin-4-yl-phenoxy)-pyrimidin-5-ylamine;
4-(2-Fluoro-4-trifluoromethyl-phenyl)-2-methyl-6-(quinolin-8-yloxy)-pyrimidin-5-ylamine;
4-(2-Fluoro-4-trifluoromethyl-phenyl)-2-methyl-6-(5,6,7,8-tetrahydro-naphthalen-2-yloxy)-pyrimidin-5-ylamine;
4-(4-Fluoro-phenoxy)-6-(2-fluoro-4-trifluoromethyl-phenyl)-2-methyl-pyrimidin-5-ylamine;
4-(Benzo[1,3]dioxol-5-yloxy)-6-(2-fluoro-4-trifluoromethyl-phenyl)-2-methyl-pyrimidin-5-ylamine;
4-(2-Chloro-4-methoxy-phenoxy)-6-(2-fluoro-4-trifluoromethyl-phenyl)-2-methyl-pyrimidin-5-ylamine;
4-(3-Fluoro-phenoxy)-6-(2-fluoro-4-trifluoromethyl-phenyl)-2-methyl-pyrimidin-5-ylamine;
4-(2-Chloro-4-trifluoromethyl-phenoxy)-6-(2-fluoro-4-trifluoromethyl-phenyl)-2-methyl-pyrimidin-5-ylamine;
4-(2-Fluoro-4-trifluoromethyl-phenyl)-6-(4-methoxy-phenoxy)-2-methyl-pyrimidin-5-ylamine;
4-(3-Chloro-5-methoxy-phenoxy)-6-(2-fluoro-4-trifluoromethyl-phenyl)-2-methyl-pyrimidin-5-ylamine;
4-(3-tert-Butyl-phenoxy)-6-(2-fluoro-4-trifluoromethyl-phenyl)-2-methyl-pyrimidin-5-ylamine;
4-(4-tert-Butyl-phenoxy)-6-(2-fluoro-4-trifluoromethyl-phenyl)-2-methyl-pyrimidin-5-ylamine;
4-(2-Fluoro-4-trifluoromethyl-phenyl)-2-methyl-6-(2-piperidin-1-yl-phenoxy)-pyrimidin-5-ylamine;
4-(2-Fluoro-phenoxy)-6-(2-fluoro-4-trifluoromethyl-phenyl)-2-methyl-pyrimidin-5-ylamine; or
4-(2-Fluoro-4-trifluoromethyl-phenyl)-2-methyl-6-phenoxy-pyrimidin-5-ylamine;
a stereoisomer thereof or a mixture of its stereoisomers, or a pharmaceutically acceptable salt thereof.

12. A pharmaceutical composition comprising a therapeutically effective amount of a 6-phenyl-pyrimidin-4-yl-(phenylamine or phenoxy) derivative of claim 1, a stereoisomer thereof or a mixture of its stereoisomers, or a pharmaceutically acceptable addition salt thereof, together with at least one pharmaceutically acceptable carrier or diluent.

13. The 6-phenyl-pyrimidin-4-yl-(phenylamine or phenoxy) derivative of claim 1, a stereoisomer thereof or a mixture of its stereoisomers, or a pharmaceutically acceptable addition salt thereof, for use as a medicament.

14-16. (canceled)

17. A method of treatment, prevention or alleviation of a disease or a disorder or a condition of a living animal body, including a human, which disorder, disease or condition is responsive to modulation of nicotinic acetylcholine receptors, which method comprises the step of administering to such a living animal body in need thereof a therapeutically effective amount of a 6-phenyl-pyrimidin-4-yl-(phenylamine or phenoxy) derivative of claim 1, a stereoisomer thereof or a mixture of its stereoisomers, or a pharmaceutically acceptable salt thereof.

18. The method according to claim 17, wherein the disease, disorder or condition responsive to modulation of nicotinic acetylcholine receptors is anxiety, a cognitive disorder, a learning deficit, a memory deficit or dysfunction, Alzheimer's disease, attention deficit, attention deficit hyperactivity disorder, Parkinson's disease, Huntington's disease, Amyotrophic Lateral Sclerosis, Gilles de la Tourette's syndrome, depression, mania, manic depression, psychosis, schizophrenia, obsessive compulsive disorders (OCD), panic disorders, an eating disorder including anorexia nervosa, bulimia and obesity, narcolepsy, nociception, AIDS-dementia, senile dementia, periferic neuropathy, autism, dyslexia, tardive dyskinesia, hyperkinesia, epilepsy, post-traumatic syndrome, social phobia, a sleeping disorder, pseudo dementia, Ganser's syndrome, pre-menstrual syndrome, late luteal phase syndrome, chronic fatigue syndrome, mutism, trichotillomania, jet-lag, hypertension, cardiac arrhythmias, a smooth muscle contraction disorder including convulsive disorders, angina pectoris, premature labour, convulsions, diarrhoea, asthma, epilepsy, tardive dyskinesia, hyperkinesia, premature ejaculation and erectile difficulty, an endocrine system disorder including thyrotoxicosis and pheochromocytoma, a neurodegenerative disorder, including transient anoxia and induced neuro-degeneration, pain, mild, moderate or severe pain, acute pain, chronic pain, pain of recurrent character, neuropathic pain, pain caused by migraine, postoperative pain, phantom limb pain, neuropathic pain, chronic headache, central pain, pain related to diabetic neuropathy, to post therapeutic neuralgia or to peripheral nerve injury, an inflammatory disorder, including an inflammatory skin disorder, acne, rosacea, Crohn's disease, inflammatory bowel disease, ulcerative colitis and diarrhoea, a disorder associated with withdrawal symptoms caused by termination of use of addictive substances, including nicotine withdrawal symptoms, opioid withdrawal symptoms, including heroin, cocaine and morphine, benzodiazepine withdrawal symptoms including benzodiazepine-like drugs and alcohol.
